SEQUENCE LISTING (6 sequences)

SEQ ID NO: 1 - DNA, 55 bp, Artificial Sequence
  misc_feature: Template
actttttactgttcgatccctgcatctcagagctcgctattccgagcttacacgt

SEQ ID NO: 2 - DNA, 13 bp, Artificial Sequence
  misc_feature: Primer
taaggctcgaatg

SEQ ID NO: 3 - DNA, 61 bp, Artificial Sequence
  misc_feature: Template
taaagctataggtacagtattagtaggacctacacctgtcaacataatggtccaggtcgtg

SEQ ID NO: 4 - DNA, 13 bp, Artificial Sequence
  misc_feature: Primer
ccaggtccagcac

SEQ ID NO: 5 - DNA, 129 bp, Artificial Sequence
  misc_feature: Template
catgtgtaacagttcctgcatgggcggcatgaacccgaggcccatcctcaccatcatcacactggaagactccagtggtaatctactgggacggaacagctttgaggtgcatgtttgtgcctgtcctgg

SEQ ID NO: 6 - DNA, 15 bp, Artificial Sequence
  misc_feature: Primer
ccaggacaggcacaa

(19) United States
(12) Patent Application Publication
(10) Pub. No.: US 2003/0027140 A1
(43) Pub. Date: Feb. 6, 2003

(54) High-fidelity DNA sequencing using solid phase capturable dideoxynucleotides and mass spectrometry

(76) Inventors: Jingyue Ju, Englewood Cliffs, NJ (US); John Robert Edwards, New York, NY (US); Zengmin Li, New York, NY (US)

Correspondence Address: John P. White, Esq., Cooper & Dunham LLP, 1185 Avenue of the Americas, New York, NY, 10036, US

(21) Appl. No.: 09/823,181

(22) Filed: Mar. 30, 2001

(51) Int. Cl. C12Q 1/68

(52) U.S. Cl. 435/6

(57) ABSTRACT

This invention provides methods for sequencing DNA by detecting the identity of a nucleotide within a DNA sequencing fragment using mass spectrometry. The invention provides cleavable linkers for attaching a label to a dideoxynucleotide and provides labeled dideoxynucleotides. The invention also provides methods for increasing mass spectrometry resolution using linkers with different mass. The invention further provides systems for separating a labeled moiety from non-labeled components in one or more samples in solution.

### BACKGROUND OF THE INVENTION

Throughout this application, various publications are referenced in parentheses by author and year. Full citations for these references may be found at the end of the specification immediately preceding the claims. The disclosures of these publications in their entireties are hereby incorporated by reference into this application to more fully describe the state of the art to which this invention pertains.

The ability to sequence deoxyribonucleic acid (DNA) accurately and rapidly is revolutionizing biology and medicine. The confluence of the massive Human Genome Project is driving an exponential growth in the development of high throughput genetic analysis technologies. This rapid technological development involving chemistry, engineering, biology, and computer science makes it possible to move from studying single genes at a time to analyzing and comparing entire genomes.

With the completion of the first entire human genome sequence map, many areas in the genome that are highly polymorphic in both exons and introns will be known. The pharmacogenomics challenge is to comprehensively identify the genes and functional polymorphisms associated with the variability in drug response (Roses, 2000). Resequencing of polymorphic areas in the genome that are linked to disease development will contribute greatly to the understanding of disease and therapeutic development. Thus, high-throughput accurate methods for resequencing the highly variable intron/exon regions of the genome are needed in order to explore the full potential of the complete human genome sequence map. The current state-of-the-art technology for high throughput DNA sequencing, such as used for the Human Genome Project (Pennisi 2000), is capillary array DNA sequencers using laser-induced fluorescence detection (Smith et al. 1986; Ju et al. 1995, 1996; Kheterpal et al. 1996; Salas-Solano et al. 1998). Improvements in the polymerases that lead to uniform termination efficiency, and the introduction of thermostable polymerases, have also significantly improved the quality of sequencing data (Tabor and Richardson, 1987, 1995).

Although this technology to some extent addresses the throughput and read length requirements of large scale DNA sequencing projects, the accuracy required for mutation studies needs to be improved for a wide variety of applications ranging from disease gene discovery to forensic identification. For example, electrophoresis based DNA sequencing methods have difficulty detecting heterozygotes unambiguously and are not 100% accurate on a given base due to compressions in regions rich in nucleotides comprising guanine (G) or cytosine (C) (Bowling et al. 1991; Yamakawa et al. 1997). In addition, the first few bases after the priming site are often masked by the high fluorescence signal from excess dye-labeled primers or dye-labeled terminators, and are therefore difficult to identify.

Mass spectrometry is able to overcome the difficulties (GC compressions and heterozygote detections) typically encountered when using capillary sequencing techniques. However, it is unable to meet the read length and throughput requirements for large scale sequencing projects. In addition, poor resolution prevents the sequence determination of large DNA fragments. At the present time, the read lengths are insufficient for de novo DNA sequencing and the stringent clean sample requirements for using mass spectrometry for. DNA sequencing are not entirely met by existing procedures. For this reason, most of the reported mass spectrometry applications have focused on single nucleotide polymorphism (SNP) detection. Several methods have been explored to this end. The most common approach is to extend a primer by a single nucleotide and detect what was added. Another technique developed by Tang et al. (1999) involves immobilizing DNA templates on a chip and again extending one base to determine a particular SNP. The same group has explored the analysis of restriction fragments to determine multiple SNPs at once (Chiu et al. 2000). Each of these techniques has been limited to analyzing only a few fragments at a time due to current limitations in mass spectra resolution. While these methods are sufficient for determining a SNP at a particular base, they require previous knowledge of the preceding sequence for primer design and synthesis. In highly variable regions of a particular gene, these methods may not suffice. Sampling only a few bases at a time could prove very inefficient.

The significant limitation to sequencing DNA with mass spectrometry is the stringent purity requirement of DNA sequencing fragments introduced to the mass spectrometer detector. DNA sequencing results have been reported by several groups using a variety of sample purification procedures. Using cleavable primers, Monforte and Becker (1997) have demonstrated read lengths up to 100 base pairs (bp). Fu et al. (1998) reported the complete sequencing of exons 5 and 3 of the p53 tumor suppressor gene using matrix assisted laser desorption/ionization time of flight (MALDI-TOF) mass spectrometry with an average read length of 35-bp. These efforts established the feasibility of using MALDI-TOF mass spectrometry for high throughput DNA sequencing up to 100-bp. In these published procedures, Monforte and Becker (1997) purified the DNA sequencing sample using a cleavable biotinylated primer, so that the extension fragments from the primer are captured by streptavidin coated magnetic beads at the 5′ end of the extension fragments, while the other components in the sequencing reaction are washed away. Fu et al. (1998) processed the sequencing samples through the use of immobilized DNA templates on a solid phase for one cycle extension. The extended DNA fragments are hybridized on the immobilized templates, while the other components in the sequencing reaction are eliminated. However, in both methods, false stopped DNA sequencing fragments are not eliminated and are introduced to the mass spectrometer. False stops occur sequencing when a deoxynucleotide rather than a dideoxynucleotide terminates a sequencing fragment. It has been shown that false stops and primers which have dimerized can produce peaks in the mass spectra that can mask the actual results preventing accurate base identification (Roskey et al. 1996).

The “lock and key” functionality of biotin and streptavidin is often utilized in biological sample preparation as a way to remove undesired impurities (Langer et al. 1981). To date these methods have involved attaching the biotin moiety on the 5′ end of the primer or the sequencing DNA template for capture by streptavidin coated magnetic beads (Tong and Smith 1992, 1993). When the samples are purified, false stops and primers that can interfere with the resulting sequencing data are not eliminated.

In addition, a further drawback of previous mass spectrometry sequencing methods was the requirement of four separate reactions, one for each dideoxynucleotide terminator analogous to the approach used in dye-labeled primer sequencing.

Ideally, for sequencing with MALDI-TOF mass spectrometry, one would like to establish a procedure that allows sequencing reactions to be performed in one tube to simplify sample preparation, to use cycle sequencing to increase the yield of the DNA sequencing fragments, and to have a method that only isolates pure DNA sequencing fragments free from false stops. The establishment of this method will form a robust procedure for sequencing DNA up to 100-bp routinely. A high fidelity DNA sequencing method has already been developed using dye-labeled primer and solid phase capturable dideoxynucleotide (ddNTP) terminators (biotinylated ddNTPs). After capture and release on the streptavidin coated solid phase, only the pure DNA sequencing fragments are loaded and detected on sequencing gels (Ju et al. 1999, 2000). This method is an effective technique to remove false stopped DNA fragments for unambiguous mutation detection of heterozygotes. However, GC rich compression issues still exist due to the use of gel electrophoresis.

To overcome the read length issue of mass spectrometry DNA sequencing, electrophore mass tags containing photo- or thermal-cleavable linkers attached to the 5′ end of DNA fragments have been explored (Xu et al. 1997, Olejnik et al. 1999). Chemical modification of DNA has been pursued with the aim of stabilizing DNA fragments as they pass through the mass spectrometer analysis process. Adding a 2′ fluoro group to the sugar moiety of the nucleotides has been shown to improve fragment stability (Ono et al. 1997). Other investigators have shown that the use of 7 deaza-purines and backbone alkylation aids in fragment stability (Schneider et al. 1995, Gut et al. 1995).

The present application discloses the use of biotinylated dideoxynucleotides for a high fidelity DNA sequencing system by mass spectrometry.

Biotinylated dideoxynucleotides and streptavidin coated magnetic beads can be used to generate high quality sequencing mass spectra of Sanger cycle sequencing DNA fragments on a MALDI-TOF mass spectrometer. The method disclosed here provides an efficient way to eliminate false stopped DNA fragments and excess primers and salts in one simple purification step, while still allowing the use of cycle sequencing to generate a high yield of sequencing fragments. Furthermore, it avoids the above-mentioned pitfalls of gel electrophoresis.

The subject application discloses that mass-tagged dideoxynucleotides which are coupled with biotin or photocleavable biotin can increase the mass separation of the DNA sequencing fragments on the mass spectra, giving better resolution than previously achievable.

Also, this application discloses a method for creating streptavidin-coated porous channels that can be used in light directed cleavage of the biotin-streptavidin complex. This is important as present commercially available streptavidin coated magnetic beads are inadequate for photocleavage purposes, in that they are opaque to ultraviolet light.

The system disclosed herein provides a high throughput and high fidelity DNA sequencing system for polymorphism and pharmacogenetics applications. Compared to gel electrophoresis sequencing, this system produces very high resolution of sequencing fragments and extremely fast separation in the time scale of microseconds. The high resolution allows accurate mutation and heterozygosity detection. Also the problematic compressions associated with gel based systems are avoided. The method disclosed here allows mass spectrometry based sequencing of much longer read lengths and higher throughput and better mass resolution than previously possible. The method also achieves the stringent sample cleaning required in mass spectrometry, eliminating false stops as well as other unnecessary components. This fast and accurate DNA resequencing system is needed in such fields as detection of single nucleotide polymorphisms (SNPs) (Chee et al. 1996), serial analysis of gene expression (Velculescu et al. 1995), identification in forensics, and genetic disease association studies.

### SUMMARY OF THE INVENTION

This invention is directed to a method for sequencing DNA by detecting the identity of a dideoxynucleotide incorporated to the 3′ end of a DNA sequencing fragment using mass spectrometry, which comprises:

(a) attaching a chemical moiety via a linker to a dideoxynucleotide to produce a labeled dideoxynucleotide;

(b) terminating a DNA sequencing reaction with the labeled dideoxynucleotide to generate a labeled DNA sequencing fragment, wherein the DNA sequencing fragment has a 3′ end and the chemical moiety is attached via the linker to the 3′ end of the DNA sequencing fragment;

(c) capturing the labeled DNA sequencing fragment on a surface coated with a compound that specifically interacts with the chemical moiety attached via the linker to the DNA sequencing fragment, thereby capturing the DNA sequencing fragment;

(d) washing the surface to remove any non-bound component;

(e) freeing the DNA sequencing fragment from the surface; and

(f) analyzing the DNA sequencing fragment using mass spectrometry so as to sequence the DNA.

This invention provides a method for sequencing DNA by detecting the identity of a plurality of dideoxynucleotides incorporated to the 3′ end of different DNA sequencing fragments using mass spectrometry, which comprises:

(a) attaching a chemical moiety via a linker to a plurality of different dideoxynucleotides to produce labeled dideoxynucleotides;

(b) terminating a DNA sequencing reaction with the labeled dideoxynucleotides to generate labeled DNA sequencing fragments, wherein the DNA sequencing fragments have a 3′ end and the chemical moiety is attached via the linker to the 3′ end of the DNA sequencing fragments;

(c) capturing the labeled DNA sequencing fragments on a surface coated with a compound that specifically interacts with the chemical moiety attached via the linker to the DNA sequencing fragments, thereby capturing the DNA sequencing fragments;

(d) washing the surface to remove any non-bound component;

(e) freeing the DNA sequencing fragments from the surface; and

(f) analyzing the DNA sequencing fragments using mass spectrometry so as to sequence the DNA.

The invention provides a linker for attaching a chemical moiety to a dideoxynucleotide, wherein the linker comprises a derivative of 4-aminomethyl benzoic acid.

The invention provides a labeled dideoxynucleotide, which comprises a chemical moiety attached via a linker to a 5-position of cytosine or thymine or to a 7-position of adenine or guanine.

The invention provides a system for separating a chemical moiety from other components in a sample in solution, which comprises:

(a) a channel coated with a compound that specifically interacts with the chemical moiety, wherein the channel comprises a plurality of ends;

(b) a plurality of wells each suitable for holding the sample;

(c) a connection between each end of the channel and a well; and

(d) a means for moving the sample through the channel between wells.

The invention provides a method of increasing mass spectrometry resolution between different DNA sequencing fragments, which comprises attaching different linkers to different dideoxynucleotides used to terminate a DNA sequencing reaction and generate different DNA sequencing fragments, wherein the different linkers increase mass separation between the different DNA sequencing fragments, thereby increasing mass spectrometry resolution.

### BRIEF DESCRIPTION OF THE FIGURES

FIG. 1: Schematic of the use of biotinylated dideoxynucleotides and a streptavidin coated solid phase to prepare DNA sequencing samples for mass spectrometric analysis. d(A, C, G, T) deoxynucleotide with base adenine (A), cytosine (C) guanine (G), or thymine (T); dd(A-b, C-b, G-b, T-b) biotinylated dideoxynucleotides.

FIG. 2: DNA sequencing data from solid phase capturable biotinylated dideoxynucleotides. The proper base is identified above each peak. The first peak is at the appropriate position and is used to identify the 13 bp primer plus the first base, adenine. The mass difference between a peak and the previous peak is indicated above the base. The region between 6500 and 12000 (m/z) is magnified for clarity. Data obtained using biotinylated dideoxynucleotides ddATP-11-biotin, ddGTP-11-biotin, ddCTP-11-biotin and ddTTP-11-biotin.

FIG. 3: Sequencing data collected using biotinylated terminators to produce sequencing fragments that are then analyzed on a mass spectrometer. All four bases can be clearly distinguished using biotinylated terminators ddATP-11-biotin, ddGTP-11-biotin, ddCTP-11-biotin and ddTTP-16-biotin.

FIG. 4: Structure of four mass tagged biotinylated ddNTPs. Any of the four ddNTPs (ddATP, ddCTP, ddGTP, ddTTP) can be used with any of the illustrated linkers.

FIG. 5: Synthesis scheme for mass tag linkers. For illustrative purposes, the linkers are labeled to correspond to the specific ddNTP with which they are shown coupled in FIGS. 4, 6, 8, 9 and 10. However, any of the three linkers can be used with any ddNTP.

FIG. 6: The synthesis of ddATP-Linker-II-11-Biotin.

FIG. 7: DNA sequencing products are purified by a streptavidin coated porous silica surface. Only the biotinylated fragments are captured. These fragments are then cleaved by ultraviolet irradiation (hv) to release the captured fragments, leaving the biotin moiety still bound to the streptavidin.

FIG. 8: Mechanism for the cleavage of photocleavable linkers.

FIG. 9: The structures of ddNTPs linked to photocleavable (PC) biotin. Any of the four ddNTPs (ddATP, ddCTP, ddGTP, ddTTP) can be used with any of the shown linkers.

FIG. 10: The synthesis of ddATP-Linker-II-PC-Biotin. PC=photocleavable.

FIG. 11: Schematic for capturing a DNA fragment terminated with a ddNTP on a surface and then for freeing the ddNTP and DNA fragment. The dideoxynucleotide (ddNTP), which is on one end of the DNA fragment (not shown), is attached via a linker to a chemical moiety “X” which interacts with a compound “Y” on the surface to capture the ddNTP and DNA fragment. The ddNTP and DNA fragment can be freed from the surface either by disrupting the interaction between chemical moiety X and compound Y (lower panel) or by cleaving a cleavable linker (upper panel).

FIG. 12: Schematic of a high throughput channel based streptavidin purification system. Sample solutions can be pushed back and forth between the two plates through glass capillaries and the streptavidin coated channels in the chip. The whole chip can be irradiated to cleave the samples after immobilization.

FIG. 13: The synthesis of streptavidin coated porous surface.

### DETAILED DESCRIPTION OF THE INVENTION

The following definitions are presented as an aid in understanding this invention.

The standard abbreviations for nucleotide bases are used as follows: adenine (A), cytosine (C), guanine (G), thymine (T), and uracil (U).

This invention is directed to a method for sequencing DNA by detecting the identity of a dideoxynucleotide incorporated to the 3′ end of a DNA sequencing fragment using mass spectrometry, which comprises:

(a) attaching a chemical moiety via a linker to a dideoxynucleotide to produce a labeled dideoxynucleotide;

(b) terminating a DNA sequencing reaction with the labeled dideoxynucleotide to generate a labeled DNA sequencing fragment, wherein the DNA sequencing fragment has a 3′ end and the chemical moiety is attached via the linker to the 3′ end of the DNA sequencing fragment;

(c) capturing the labeled DNA sequencing fragment on a surface coated with a compound that specifically interacts with the chemical moiety attached via the linker to the DNA sequencing fragment, thereby capturing the DNA sequencing fragment;

(d) washing the surface to remove any non-bound component;

(e) freeing the DNA sequencing fragment from the surface; and

(f) analyzing the DNA sequencing fragment using mass spectrometry so as to sequence the DNA.

This invention provides a method for sequencing DNA by detecting the identity of a plurality of dideoxynucleotides incorporated to the 3′ end of different DNA sequencing fragments using mass spectrometry, which comprises:

(a) attaching a chemical moiety via a linker to a plurality of different dideoxynucleotides to produce labeled dideoxynucleotides;

(b) terminating a DNA sequencing reaction with the labeled dideoxynucleotides to generate labeled DNA sequencing fragments, wherein the DNA sequencing fragments have a 3′ end and the chemical moiety is attached via the linker to the 3′ end of the DNA sequencing fragments;

(c) capturing the labeled DNA sequencing fragments on a surface coated with a compound that specifically interacts with the chemical moiety attached via the linker to the DNA sequencing fragments, thereby capturing the DNA sequencing fragments;

(d) washing the surface to remove any non-bound component;

(e) freeing the DNA sequencing fragments from the surface; and

(f) analyzing the DNA sequencing fragments using mass spectrometry so as to sequence the DNA.

In one embodiment, the chemical moiety is attached via a different linker to different dideoxynucleotides. In one embodiment, the different linkers increase mass separation between different labeled DNA sequencing fragments and thereby increase mass spectrometry resolution.

In one embodiment, the dideoxynucleotide is selected from the group consisting of 2′,3′-dideoxyadenosine 5′-triphosphate (ddATP), 2′,3′-dideoxyguanosine 5′-triphosphate (ddGTP), 2′,3′-dideoxycytidine 5′-triphosphate (ddCTP), and 2′,3′-dideoxythymidine 5′-triphosphate (ddTTP).

In different embodiments of the methods described herein, the interaction between the chemical moiety attached via the linker to the DNA sequencing fragment and the compound on the surface comprises a biotin-streptavidin interaction, a phenylboronic acid-salicylhydroxamic acid interaction, or an antigen-antibody interaction.

In one embodiment, the step of freeing the DNA sequencing fragment from the surface comprises disrupting the interaction between the chemical moiety attached via the linker to the DNA sequencing fragment and the compound on the surface. In different embodiments, the interaction is disrupted by a means selected from the group consisting of one or more of a physical means, a chemical means, a physical chemical means, heat, and light. In one embodiment, the interaction is disrupted by ultraviolet light. In different embodiments, the interaction is disrupted by ammonium hydroxide, formamide, or a change in pH (−log H⁺ concentration) In different embodiments, the linker can comprise a chain structure, or a structure comprising one or more rings, or a structure comprising a chain and one or more rings. In different embodiments, the dideoxynucleotide comprises a cytosine or a thymine with a 5-position, or an adenine or a guanine with a 7-position, and the linker is attached to the 5-position of cytosine or thymine or to the 7-position of adenine or guanine.

In one embodiment, the step of freeing the DNA sequencing fragment from the surface comprises cleaving the linker. In different embodiments, the linker is cleaved by a means selected from the group consisting of one or more of a physical means, a chemical means, a physical chemical means, heat; and light. In one embodiment, the linker is cleaved by ultraviolet light. In different embodiments, the linker is cleaved by ammonium hydroxide, formamide, or a change in pH (−log H⁺ concentration).

In one embodiment, the linker comprises a derivative of 4-aminomethyl benzoic acid. In one embodiment, the linker comprises one or more fluorine atoms.

In one embodiment, the linker is selected from the group consisting of:

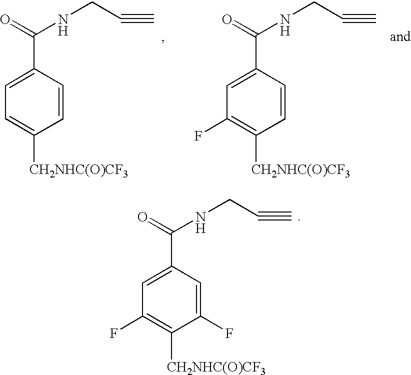

In one embodiment, a plurality of different labeled dideoxynucleotides is used to generate a plurality of different labeled DNA sequencing fragments. In one embodiment, a plurality of different linkers is used to increase mass separation between different labeled DNA sequencing fragments and thereby increase mass spectrometry resolution.

In one embodiment, the chemical moiety comprises biotin, the labeled dideoxynucleotide is a biotinylated dideoxynucleotide, the labeled DNA sequencing fragment is a biotinylated DNA sequencing fragment, and the surface is a streptavidin-coated solid surface. In one embodiment, the biotinylated dideoxynucleotide is selected from the group consisting of ddATP-11-biotin, ddCTP-11-biotin, ddGTP-11-biotin, and ddTTP-16-biotin.

In one embodiment, the biotinylated dideoxynucleotide is selected from the group consisting of:

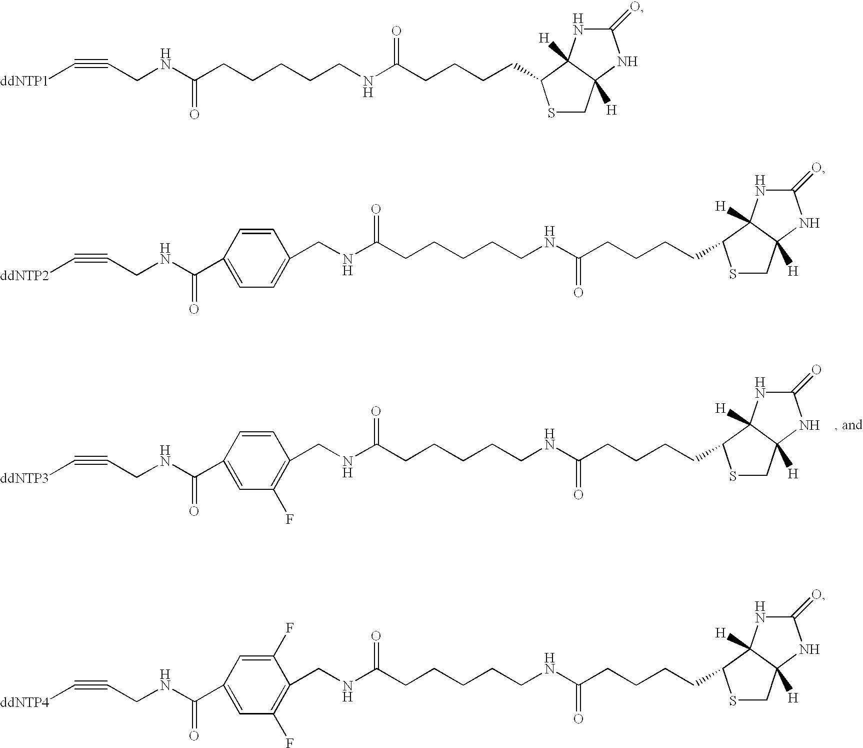

wherein ddNTP1, ddNTP2, ddNTP3, and ddNTP4 represent four different dideoxynucleotides.

In one embodiment, the biotinylated dideoxynucleotide is selected from the group consisting of:

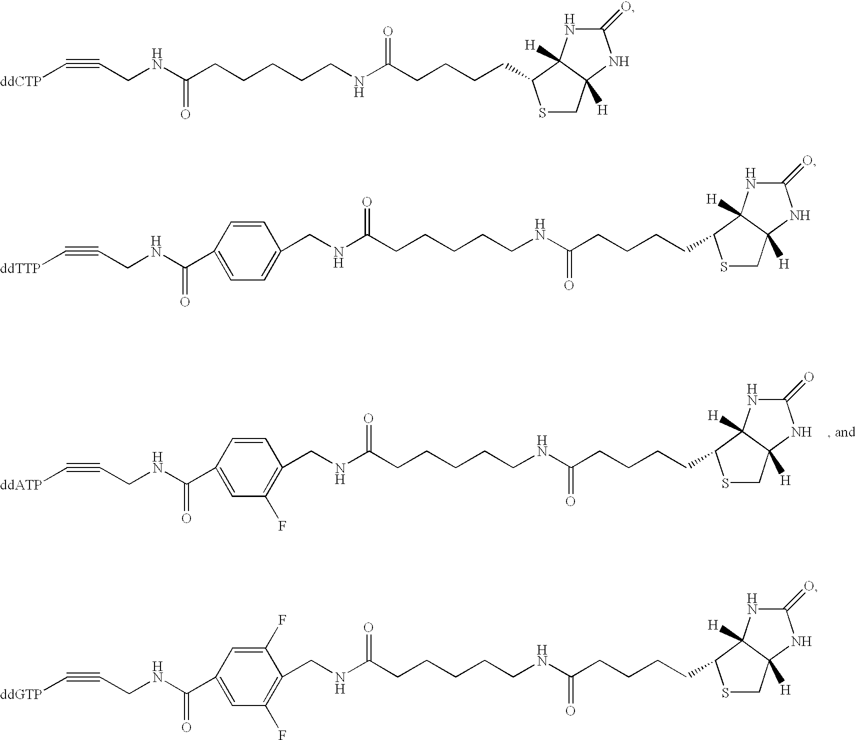

In one embodiment, the biotinylated dideoxynucleotide is selected from the group consisting of:

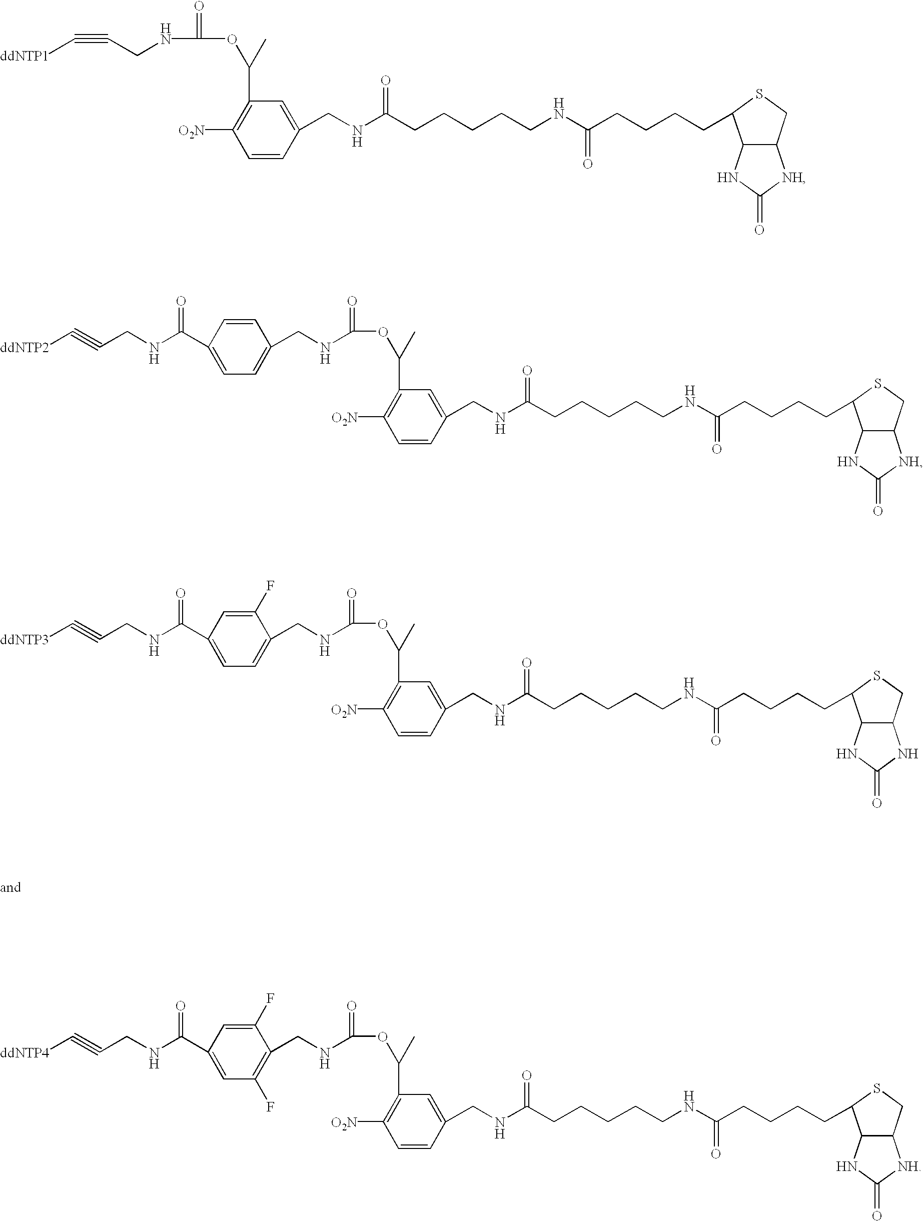

wherein ddNTP1, ddNTP2, ddNTP3, and ddNTP4 represent four different dideoxynucleotides.

In one embodiment, the biotinylated dideoxynucleotide is selected from the group consisting of:

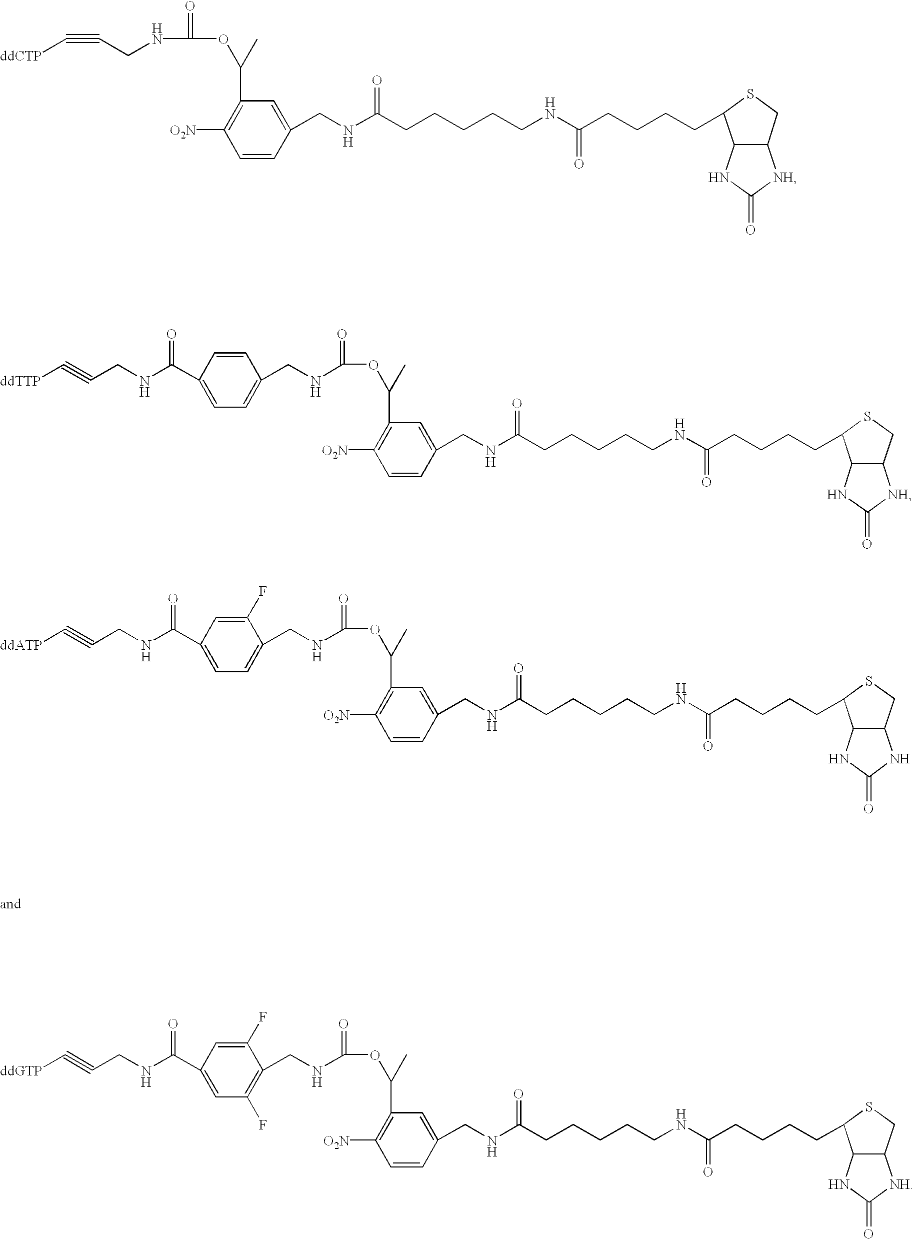

In one embodiment, the streptavidin-coated solid surface is a streptavidin-coated magnetic bead or a streptavidin-coated silica glass.

In one embodiment of the method, steps (b) to (e) are performed in a single container or in a plurality of connected containers.

In one embodiment, the mass spectrometry is matrix-assisted laser desorption/ionization time-of-flight mass spectrometry.

The invention provides for the use of any of the methods described herein for detection of single nucleotide polymorphisms, genetic mutation analysis, serial analysis of gene expression, gene expression analysis, identification in forensics, genetic disease association studies, genomic sequencing, translational analysis, or transcriptional analysis.

The invention provides a linker for attaching a chemical moiety to a dideoxynucleotide, wherein the linker comprises a derivative of 4-aminomethyl benzoic acid.

In one embodiment, the dideoxynucleotide is selected from the group consisting of 2′,3′-dideoxyadenosine 5′-triphosphate (ddATP), 2′,3′-dideoxyguanosine 5′-triphosphate (ddGTP), 2′,3′-dideoxycytidine 5′-triphosphate (ddCTP), and 2′,3′-dideoxythymidine 5′-triphosphate (ddTTP).

In one embodiment, the linker comprises one or more fluorine atoms.

In one embodiment, the linker is selected from the group consisting of:

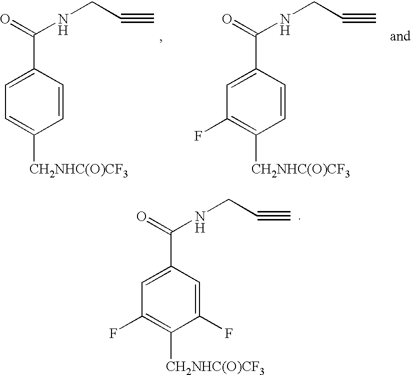

In different embodiments, the linker can comprise a chain structure, or a structure comprising one or more rings, or a structure comprising a chain and one or more rings.

In different embodiments, the linker is cleavable by a means selected from the group consisting of one or more of a physical means, a chemical means, a physical chemical means, heat, and light. In one embodiment, the linker is cleavable by ultraviolet light. In different embodiments, the linker is cleavable by ammonium hydroxide, formamide, or a change in pH (−log H⁺ concentration).

In different embodiments of the linker, the chemical moiety comprises biotin, streptavidin, phenylboronic acid, salicylhydroxamic acid, an antibody, or an antigen.

In different embodiments, the dideoxynucleotide comprises a cytosine or a thymine with a 5-position, or an adenine or a guanine with a 7-position, and the linker is attached to the 5-position of cytosine or thymine or to the 7-position of adenine or guanine.

The invention provides for the use of any of the linkers described herein in DNA sequencing using mass spectrometry, wherein the linker increases mass separation between different dideoxynucleotides and increases mass spectrometry resolution.

The invention provides a labeled dideoxynucleotide, which comprises a chemical moiety attached via a linker to a 5-position of cytosine or thymine or to a 7-position of adenine or guanine.

In one embodiment, the dideoxynucleotide is selected from the group consisting of 2′,3′-dideoxyadenosine 5′-triphosphate (ddATP), 2′,3′-dideoxyguanosine 5′-triphosphate (ddGTP), 2′,3′-dideoxycytidine 5′-triphosphate (ddCTP), and 2′,3′-dideoxythymidine 5′-triphosphate (ddTTP).

In different embodiments, the linker can comprise a chain structure, or a structure comprising one or more rings, or a structure comprising a chain and one or more rings. In different embodiments, the linker is cleavable by a means selected from the group consisting of one or more of a physical means, a chemical means, a physical chemical means, heat, and light. In one embodiment, the linker is cleavable by ultraviolet light. In different embodiments, the linker is cleavable by ammonium hydroxide, formamide, or a change in pH (−log H⁺ concentration)

In different embodiments of the labeled dideoxynucleotide, the chemical moiety comprises biotin, streptavidin, phenylboronic acid, salicylhydroxamic acid, an antibody, or an antigen.

In one embodiment, the labeled dideoxynucleotide is selected from the group consisting of:

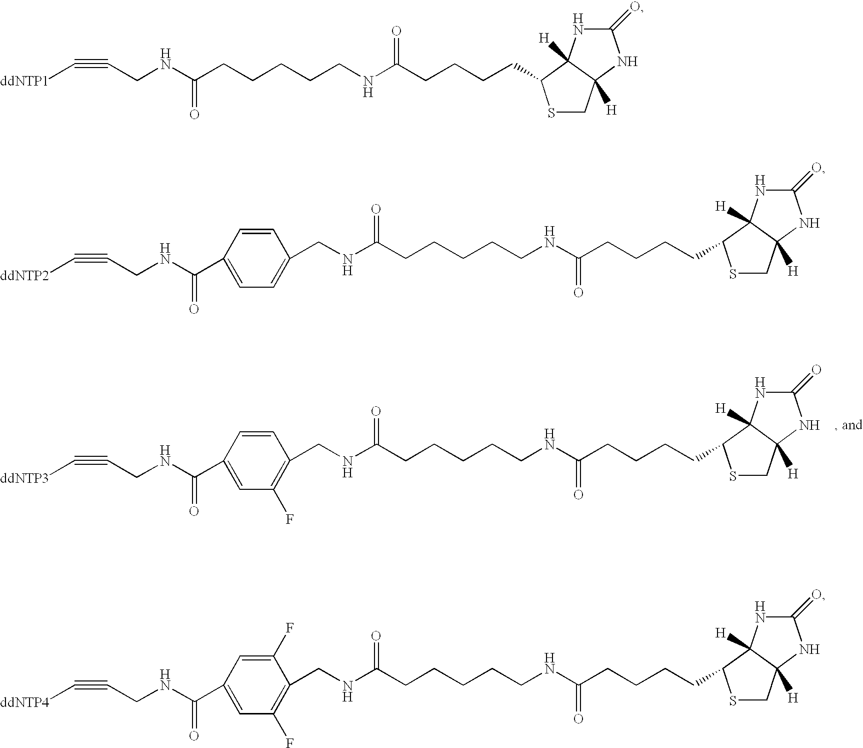

wherein ddNTP1, ddNTP2, ddNTP3, and ddNTP4 represent four different dideoxynucleotides.

In one embodiment, the labeled dideoxynucleotide is selected from the group consisting of:

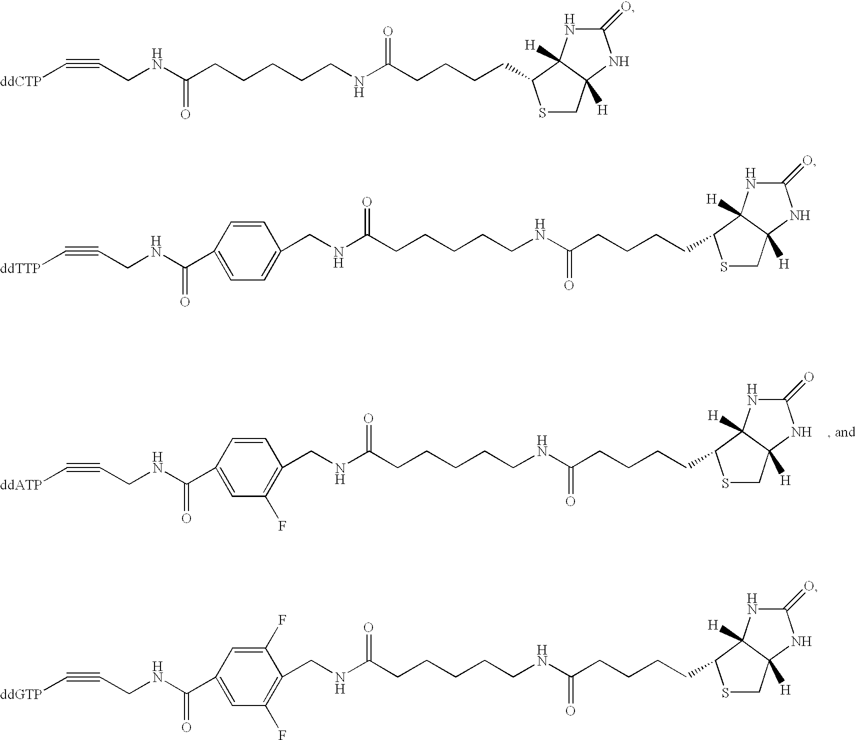

In one embodiment, the labeled dideoxynucleotide is selected from the group consisting of:

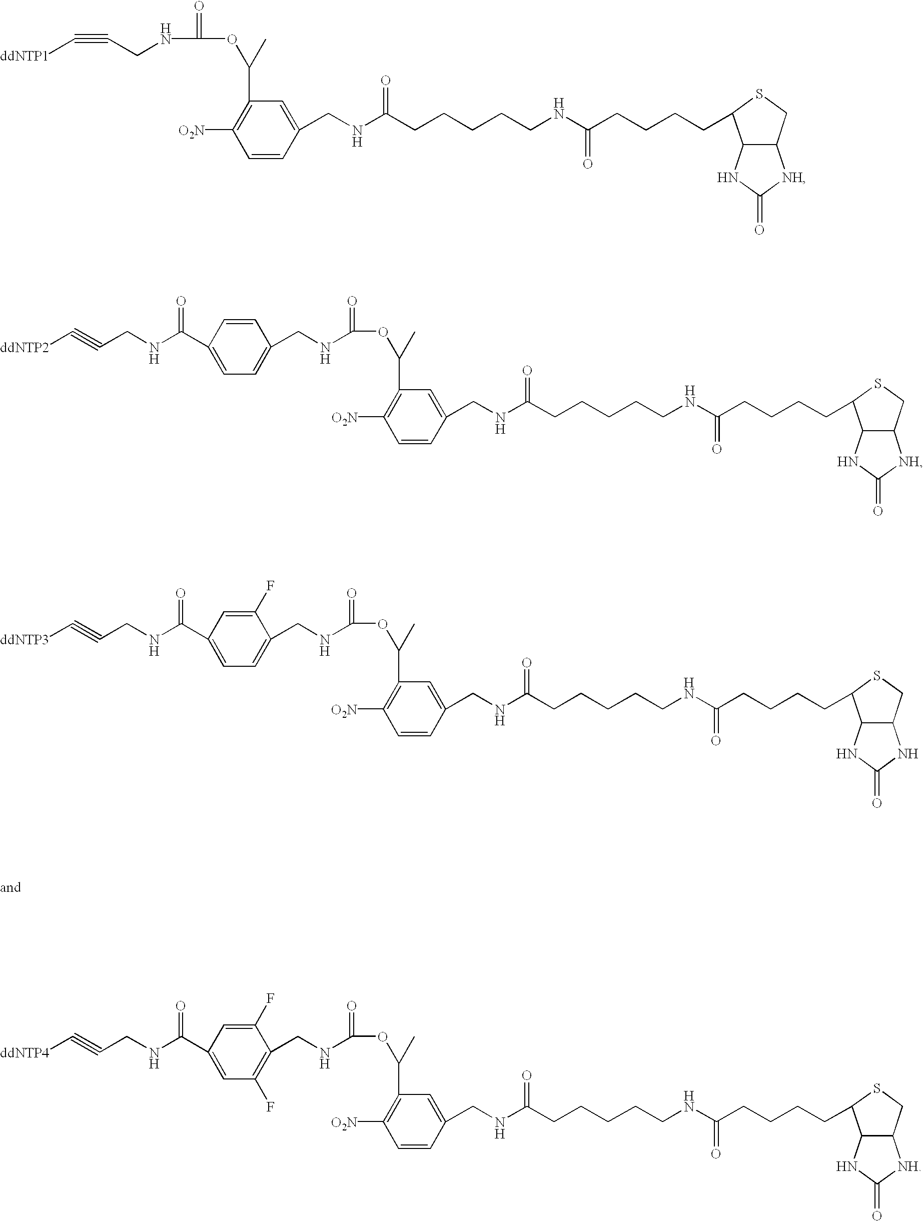

wherein ddNTP1, ddNTP2, ddNTP3, and ddNTP4 represent four different dideoxynucleotides.

In one embodiment, the labeled dideoxynucleotide is selected from the group consisting of:

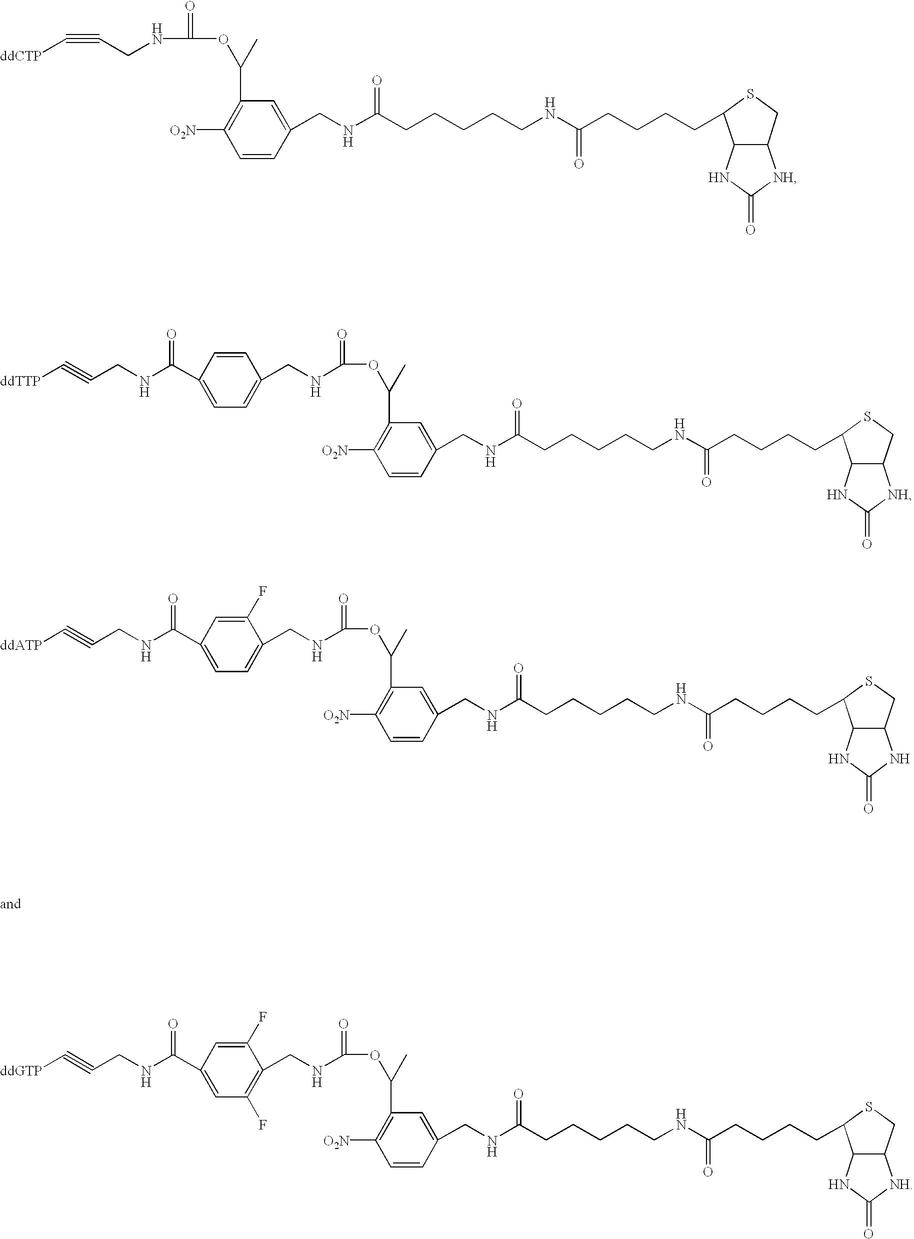

The invention provides the use of any of the labeled dideoxynucleotide described herein in DNA sequencing using mass spectrometry, wherein the linker increases mass separation between different labeled dideoxynucleotides and increases mass spectrometry resolution.

In one embodiment, the labeled dideoxynucleotide has a molecular weight selected from the group consisting of 844, 977, 1,017, and 1,051. In one embodiment, the labeled dideoxynucleotide has a molecular weight selected from the group consisting of 1,049, 1,182, 1,222, and 1,257.

In one embodiment the mass spectrometry is matrix-assisted laser desorption/ionization time-of-flight mass spectrometry.

The invention provides a system for separating a chemical moiety from other components in a sample in solution, which comprises:

(a) a channel coated with a compound that specifically interacts with the chemical moiety, wherein the channel comprises a plurality of ends;

(b) a plurality of wells each suitable for holding the sample;

(c) a connection between each end of the channel and a well; and

(d) a means for moving the sample through the channel between wells.

In one embodiment of the system, the interaction between the chemical moiety and the compound coating the surface is a biotin-streptavidin interaction, a phenylboronic acid-salicylhydroxamic acid interaction, or an antigen-antibody interaction.

In one embodiment, the chemical moiety is a biotinylated moiety and the channel is a streptavidin-coated silica glass channel. In one embodiment, the biotinylated moiety is a biotinylated DNA sequencing fragment.

In one embodiment, the chemical moiety can be freed from the surface by disrupting the interaction between the chemical moiety and the compound coating the surface. In different embodiments, the interaction can be disrupted by a means selected from the group consisting of one or more of a physical means, a chemical means, a physical chemical means, heat, and light. In different embodiments, the interaction can be disrupted by ammonium hydroxide, formamide, or a change in pH (−log H⁺ concentration).

In one embodiment, the chemical moiety is attached via a linker to another chemical compound. In one embodiment, the other chemical compound is a DNA sequencing fragment. In one embodiment, the linker is cleavable by a means selected from the group consisting of one or more of a physical means, a chemical means, a physical chemical means, heat, and light. In one embodiment, the channel is transparent to ultraviolet light and the linker is cleavable by ultraviolet light. Cleaving the linker frees the DNA sequencing fragment or other chemical compound from the chemical moiety which remains captured on the surface.

The invention provides a multi-channel system which comprises a plurality of any of the single channel systems disclosed herein. In one embodiment, the channels are in a chip. In one embodiment, the multi-channel system comprises 96 channels in a chip.

The invention provides for the use of any of the systems described herein for separating one or more DNA sequencing fragments, wherein each fragment is terminated with a dideoxynucleotide attached via a linker to the chemical moiety.

The invention provides a method of increasing mass spectrometry resolution between different DNA sequencing fragments, which comprises attaching different linkers to different dideoxynucleotides used to terminate a DNA sequencing reaction and generate different DNA sequencing fragments, wherein the different linkers increase mass separation between the different DNA sequencing fragments, thereby increasing mass spectrometry resolution.

In one embodiment, one or more of the different linkers comprises one or more fluorine atoms.

In one embodiment, one or more of the different linkers is selected from the group consisting of:

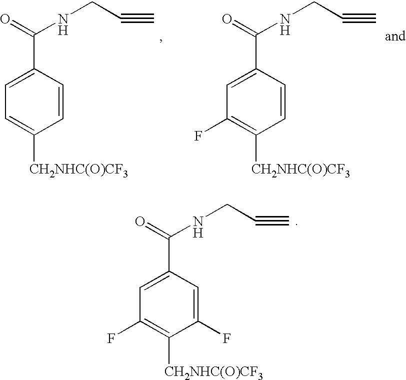

This invention will be better understood from the Experimental Details which follow. However, one skilled in the art will readily appreciate that the specific methods and results discussed are merely illustrative of the invention as described more fully in the claims which follow thereafter.

Experimental Details

I. DNA Sequencing with Biotinylated Dideoxynucleotides on a Mass Spectrometer

Matrix-assisted laser desorption/ionization time-of-flight mass spectrometry (MALDI-TOF MS) has recently been explored widely for DNA sequencing. The Sanger dideoxy procedure (Sanger et al. 1977) is used to generate the DNA sequencing fragments and no labels are required. The mass resolution in theory can be as good as one dalton. Thus, compared to gel electrophoresis sequencing systems, mass spectrometry produces very high resolution of the sequencing fragments and extremely fast separation in the time scale of microseconds. The high resolution allows accurate mutation and heterozygosity detection. Another advantage of sequencing with mass spectrometry is that the compressions associated with gel based systems are completely eliminated. However, in order to obtain accurate measure of the mass of the sequencing DNA fragments, the samples must be free from alkaline and alkaline-earth salts. Samples must be desalted and free from contaminants before the MS analysis.

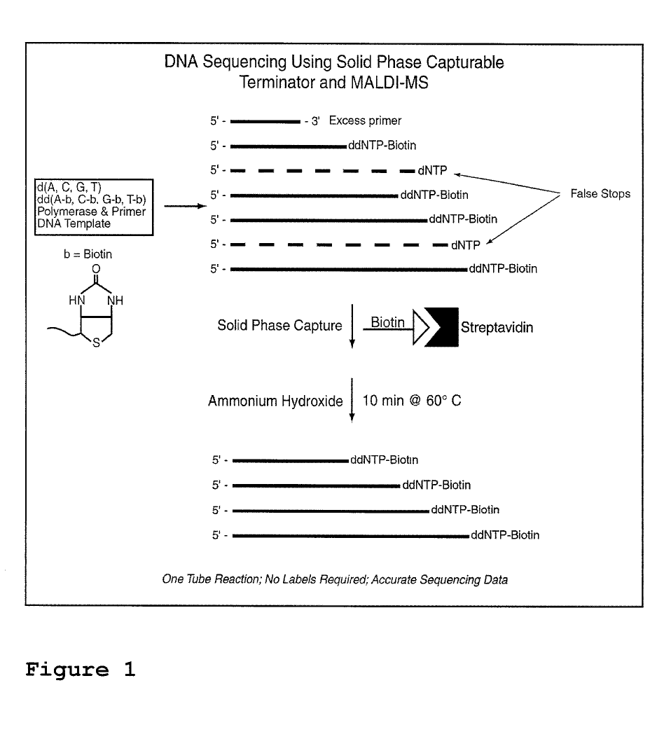

A general scheme to meet all these requirement for preparing DNA sequencing fragments using biotinylated dideoxynucleotides and streptavidin coated solid phase is shown in FIG. 1. In different embodiments of the methods described herein, affinity systems other than biotin-streptavidin can be used. Such affinity systems include but are not limited to phenylboronic acid-salicylhydroxamic acid (Bergseid et al. 2000) and antigen-antibody systems.

As illustrated schematically in FIG. 1, DNA template, deoxynucleotides (dNTPs) (A, C, G, T) and biotinylated dideoxynucleotides (ddNTP-biotin) (A-b, C-b, G-b, T-b), primer, and DNA polymerase are combined in one tube. After polymerase extension and termination reactions, a series of DNA sequencing fragments with different lengths are generated. The sequencing reaction mixture is then incubated for a few minutes with a streptavidin coated solid phase. Only the DNA sequencing fragments that are terminated with biotinylated dideoxynucleotide at the 3′ end are captured on the solid phase. Excess primers, false terminated DNA fragments (fragments terminated at dNTPs instead of ddNTPs), enzymes and all other components from the sequencing reaction are washed away. The biotinylated DNA sequencing fragments are then cleaved off the solid phase by disrupting the interaction between biotin and streptavidin to obtain a pure set of DNA sequencing fragments. The interaction between biotin and streptavidin can be disrupted using, for example, ammonium hydroxide, formamide, or a change in pH. The DNA sequencing fragments are then mixed with matrix (3-hydroxy-picolinic acid) and loaded into a mass spectrometer to produce accurate mass spectra of the DNA sequencing fragments. Since each type of nucleotide has a unique molecular mass, the mass difference between adjacent peaks on the mass spectra gives the sequence identity of the nucleotides.

In DNA sequencing with mass spectrometry, the purity of the samples directly affects the quality of the obtained spectra. Excess primers, salts, and fragments that are prematurely terminated in the sequencing reactions (false stops) will create extra noise and extraneous peaks (Fu et al. 1998). Excess primers can also dimerize to form high molecular weight species that give a false signal in mass spectrometry (Wu et al. 1993). False stops occur in sequencing when a deoxynucleotide rather than a dideoxynucleotide terminates a sequencing fragment. A deoxynucleotide terminated false stop has a mass difference of 16 daltons with its dieoxy counterpart. This mass difference is identical to the difference between adenine and guanine. Thus, false stops can be wrongly interpreted or interfere with existing peaks decreasing accuracy. Salts can ruin spectra by broadening the observed peaks beyond recognition. The method disclosed here eliminates all these problems.

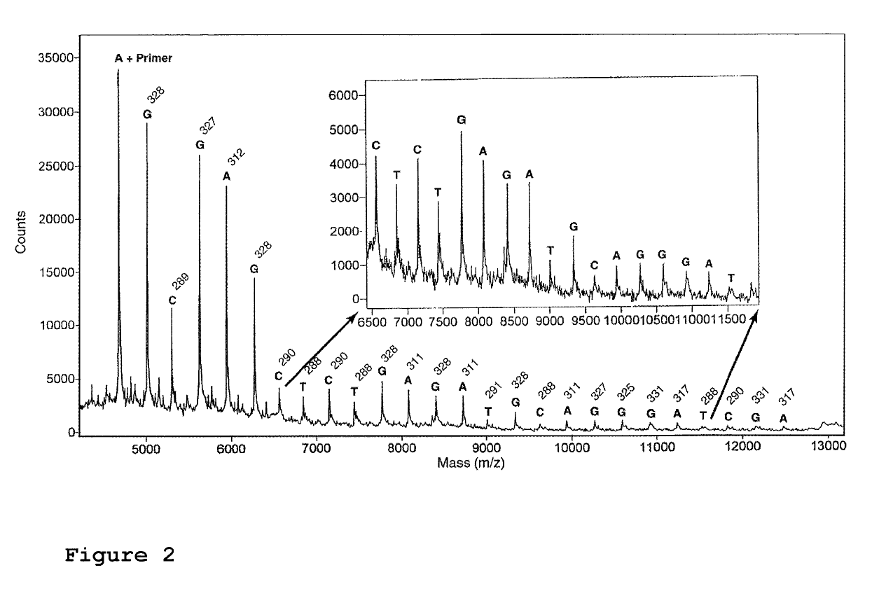

Previously, Ju et al. (1999, 2000) established a procedure for accurately sequencing DNA using fluorescent dye-labeled primer and biotinylated dideoxynucleotides. Upon capture and release from streptavidin-coated magnetic beads, all the falsely stopped fragments are completely removed. This application discloses a method to obtain sequencing data using biotinylated dideoxynucleotides (strategy shown in FIG. 1) with MALDI-TOF mass spectrometry as shown in FIG. 2. The sequencing data in FIG. 2 were generated using the following 55 bp synthetic template (SEQ ID NO: 1) and 13 bp primer (SEQ ID NO: 2):

```
5′-ACTTTTTACTGTTCGATCCCTGCATCTCAGAGCTCGCTATTCCGAGCTTACACGT-3′
                Template  |||||||||||||
                                                                            3′-TAAGGCTCGAATG-5′
                                                                                        Primer
```

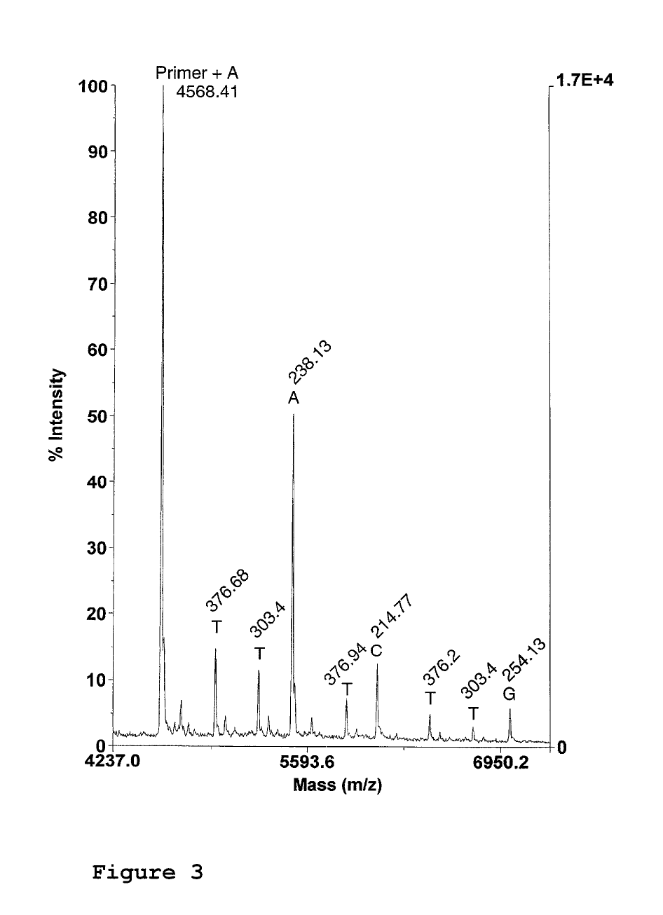

Four commercially available biotinylated dideoxynucleotides ddATP-11-biotin, ddGTP-11-biotin, ddCTP-11-biotin and ddTTP-11-biotin (New England Nuclear, Boston) were used to produce the sequencing ladder that was generated all in one tube using the cycle sequencing procedure. It can be seen from FIG. 2 that very clean sequence peaks are obtained on the mass spectra, with the first peak being primer extended by one biotinylated dideoxynucleotide. Furthermore, excess primer in the sequencing reaction is completely removed and no false stopped peaks are detected. The base identity of A and G can be identified unambiguously in FIG. 2. Since the mass difference between the commercially available ddCTP-11-Biotin and ddTTP-11-biotin is one dalton and the resolution is only within about 3 daltons in the mass detector for DNA fragments, C and T cannot be differentiated in FIG. 2. The data shows that by capturing/releasing DNA sequencing fragments with the biotin located on the 3′ dideoxy terminators, clean sequencing ladders that are free from any other contaminants can be obtained. Further improvement of the procedure requires the use of biotinylated ddTTPs that have large mass differences in comparison to ddCTP-11-biotin. To achieve this, ddTTP-16-biotin is used since it is commercially available (Enzo, Boston) and has a large mass difference in comparison to ddCTP-11-biotin (see Table 1). It is paired with ddCTP-ll-biotin, ddATP-11-biotin, and ddGTP-11-biotin to allow unambiguous assignment of the mass spectra sequencing ladder (see FIG. 3).

TABLE 1

| | Normal | Commercial | Biotinylated ddNTP |
|---|---|---|---|
| Base | ddNTP | Biotinylated ddNTP | with mass tag linker |
| C relative to C | 0 | 0 | 0 (no extra linker) |
| T relative to C | 15 | 88.5 (16 linker) | 125 (Linker I) |
| A relative to C | 24 | 24 | 165 (Linker II) |
| G relative to C | 40 | 40 | 200 (Linker III) |
| Smallest relative | 9 | 16 | 35 |
| difference | | | |

Relative mass differences of dideoxynucleotides using ddCTP as a reference. The relative difference between a fragment and one additional base is about 300 daltons. All relative masses are in daltons.

Sample preparation is performed in one tube by executing the sequencing reactions with biotinylated ddNTPs, regular dNTPs, DNA polymerase, and reaction buffer. The sample is then placed in a thermocycler for 30 cycles to create extension fragments. Streptavidin beads are then added to the sample and incubated to allow the biotin-streptavidin complex to form. The beads are collected by placing the reaction tube in a magnet and thoroughly washing them with an ammonium acetate solution to remove all impurities such as false stops, primers, and salts. Dilute ammonium hydroxide solution is then used to dissociate the biotin streptavidin complex at 60° C. (Jurinke et. al., 1997). Once this complex is dissociated, the solution is placed back in the magnet to separate the beads out of solution. The supernatant is collected, added to a matrix solution of 3-hydroxy-picolinic acid (Aldrich), and allowed to crystallize for analysis by a Perkin Elmer Voyager DE MALDI-TOF mass spectrometer. The resulting spectrum is assigned according to the positions of the various peaks.

II. Design and Synthesis of Biotinylated Dideoxynucleotides with Mass Tags

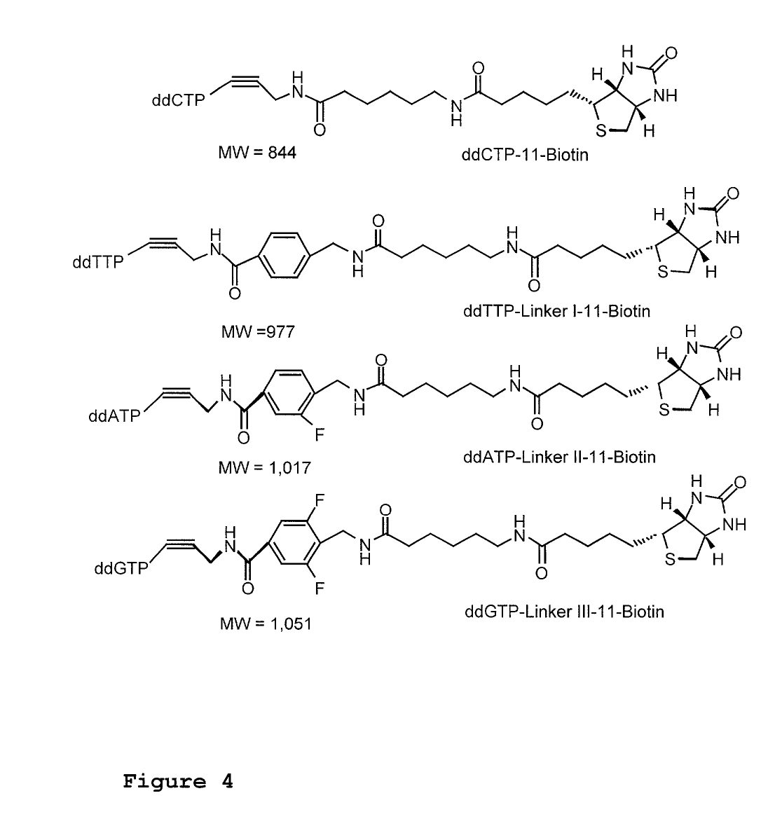

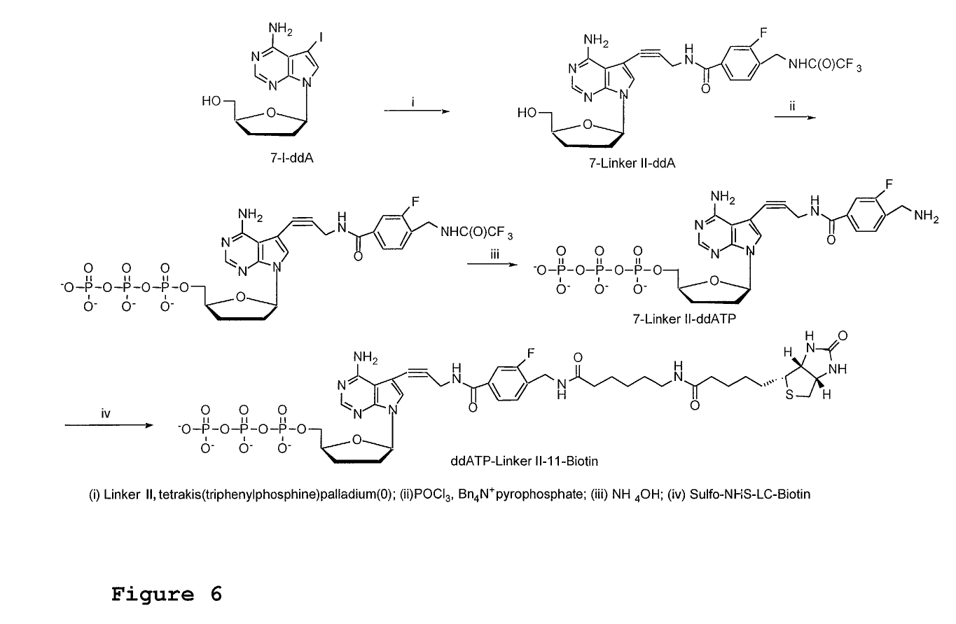

The ability to distinguish various bases in DNA using mass spectrometry is dependent on the mass differences of the bases in the spectra. For the above work, the smallest difference mass between any two nucleotides is 16 daltons (see Table 1). Fei et al. (1988) realized this problem and have shown that using dye-labeled ddNTP paired with a regular dNTP to space out the mass difference, an increase in the detection resolution in a single nucleotide extension assay can be achieved. To enhance the ability to distinguish peaks in sequencing spectra, the current application discloses systematic modification of the biotinylated dideoxynucleotides by incorporating mass linkers assembled using 4-aminomethyl benzoic acid derivatives to increase the mass separation of the individual bases. The mass linkers can be modified by incorporating one or two fluorine atoms to further space out the mass differences between the nucleotides. The structures of four biotinylated ddNTPs are shown in FIG. 4. ddCTP-11-biotin is commercially available (New England Nuclear, Boston). ddTTP-Linker I-11-Biotin, ddATP-Linker II-11-Biotin and ddGTP-Linker III-11-Biotin are synthesized as shown, for example, for ddATP-Linker II-11-Biotin in FIG. 6. In designing these mass tag linker modified biotinylated ddNTPs, the linkers are attached to the 5-position on the pyrimidine bases (C and T), and to the 7-position on the purines (A and G) for subsequent conjugation with biotin. It has been established that modification of these positions on the bases in the nucleotides, even with bulky energy transfer fluorescent dyes, still allows efficient incorporation of the modified nucleotides into the DNA strand by DNA polymerase (Rosenblum et al. 1997, Zhu et al. 1994). Thus, the ddNTPs-Linker-11-biotin can be incorporated into the growing strand by the polymerase in DNA sequencing reactions.

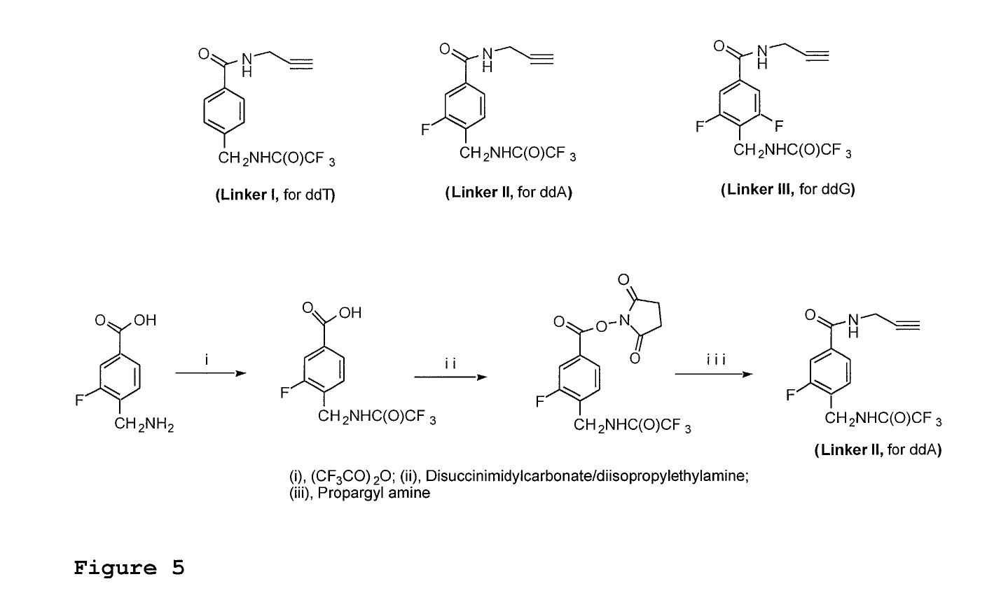

Larger mass separations will greatly aid in longer read lengths where signal intensity is smaller and resolution is lower. The smallest mass difference between two individual bases is over three times as great in the mass tagged biotinylated ddNTPs compared to normal ddNTPs and more than double that achieved by the standard biotinylated ddNTPs as shown in Table 1. Three 4-aminomethyl benzoic acid derivatives Linker I, Linker II and Linker III are designed as mass tags as well as linkers for bridging biotin to the corresponding dideoxynucleotides. The synthesis of Linker II (FIG. 5) is described here to illustrate the synthetic procedure. 3-Fluoro-4-aminomethyl benzoic acid that can be easily prepared via published procedures (Maudling et al. 1983; Rolla 1982) is first protected with trifluoroacetic anhydride, then converted to N-hydroxysuccinimide (NHS) ester with disuccinimidylcarbonate in the presence of diisopropylethylamine. The resulting NHS ester is subsequently coupled with commercially available propargylamine to form the desired compound, Linker II. Using an analogous procedure, Linker I and Linker III can be easily constructed.

FIG. 6 describes the scheme required to prepare biotinylated ddATP-Linker II-11-Biotin using well-established procedures (Prober et al. 1987; Lee et al. 1992; Hobbs et al. 1991). 7-1-ddA is coupled with linker II in the presence of tetrakis (triphenylphosphine) palladium(0) to produce 7-Linker II-ddA, which is phosphorylated with POCl₃ in butylammonium pyrophosphate (Burgess and Cook, 2000). After removing the trifluoroacetyl group with ammonium hydroxide, 7-Linker II-ddATP is produced, which then couples with sulfo-NHS-LC-Biotin (Pierce, Rockford Ill.) to yield the desired ddATP-Linker II-11-Biotin. Similarly, ddTTP-Linker I-11-Biotin, and ddGTP-Linker III-11-Biotin can be synthesized.

III. Design and Synthesis of Mass Tagged ddNTPs Containing Photocleavable Biotin for a High Fidelity and High Throughput DNA Sequencing System using Mass Spectrometry

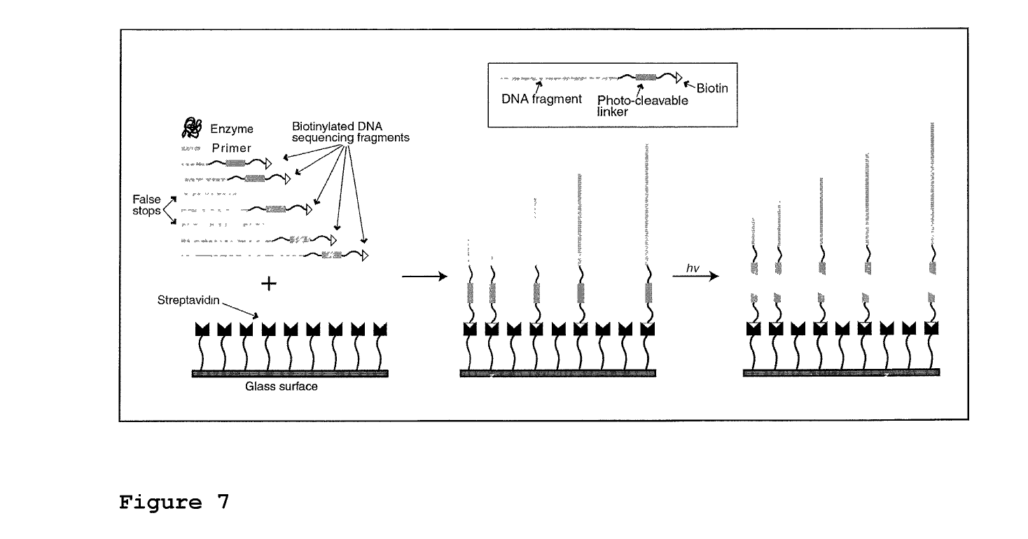

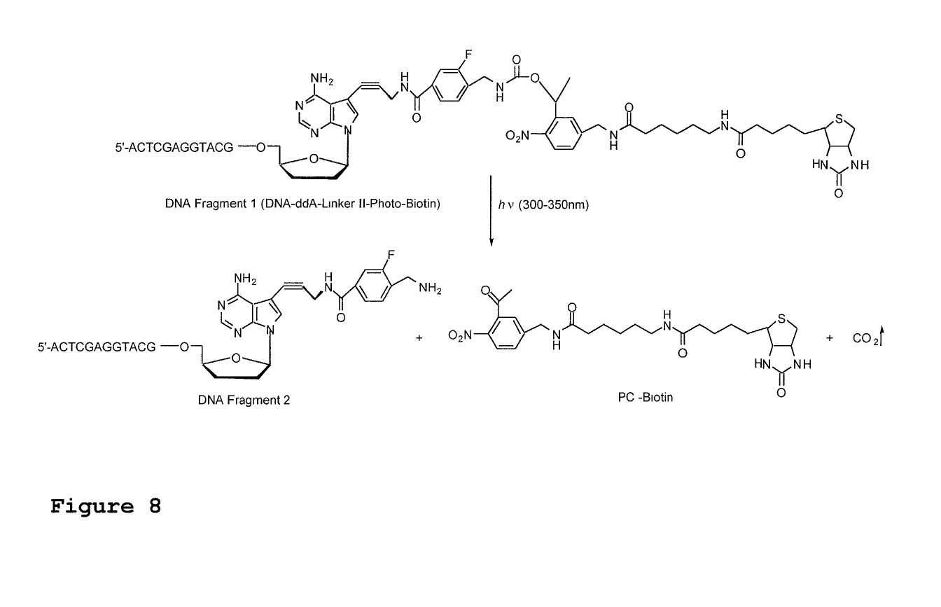

To further optimize the sequencing system this application discloses the use of ddNTPs containing a photocleavable biotin (PC-biotin). A schematic of capture and cleavage of the photocleavable linker on the streptavidin coated porous surface is shown in FIG. 7. At the end of DNA sequencing reaction, the reaction mixture consists of excess primers, enzymes, salts, false stops, and the desired sequencing fragments. This reaction mixture is passed over a streptavidin-coated surface and allowed to incubate. The biotinylated sequencing fragments are captured by the streptavidin surface, while everything else in the mixture is washed away. Then the fragments are released into solution by cleaving the photocleavable linker with ultraviolet (UV) light, while the biotin remains attached to the streptavidin that is covalently bound to the surface. The pure DNA fragments can then be crystallized in matrix solution and analyzed by mass spectrometry. It is advantageous to cleave the biotin moiety since it contains sulfur which has several relatively abundant isotopes. The rest of the DNA fragments and linkers contain only carbon, nitrogen, hydrogen, oxygen, fluorine and phosphorous, whose dominant isotopes are found with a relative abundance of 99% to 100%. This allows high resolution mass spectra to be obtained. The photocleavage mechanism (Olejnik et al. 1995, 1999) is shown in FIG. 8. Upon irradiation with ultraviolet light at 300-350 nm, the light sensitive o-nitroaromatic carbonamide functionality on DNA fragment 1 is cleaved, producing DNA fragment 2, PC-biotin and carbon dioxide. The partial chemical linker remaining on DNA fragment 2 is stable for detection by mass spectrometry.

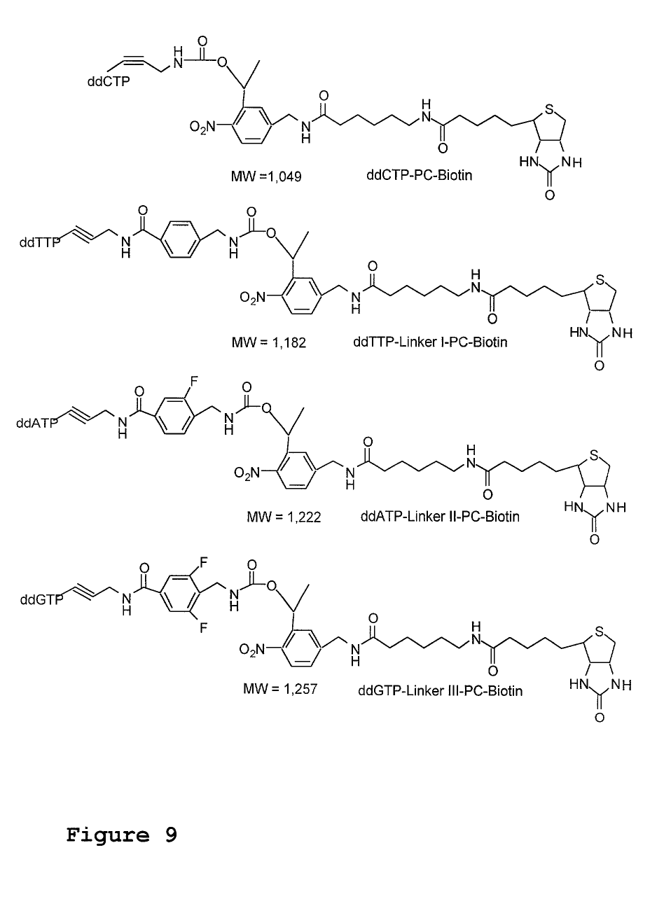

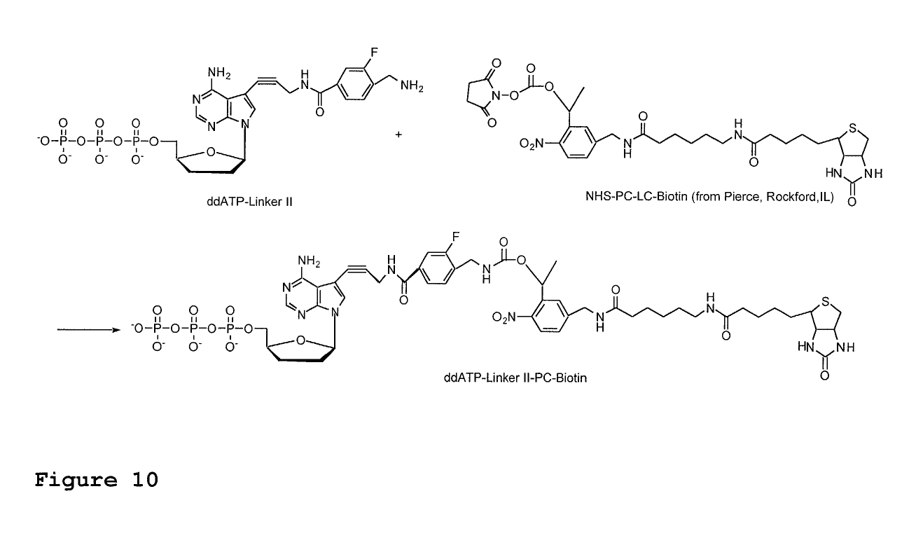

Four new biotinylated ddNTPs disclosed here, ddCTP-PC-Biotin, ddTTP-Linker I-PC-Biotin, ddATP-Linker II-PC-Biotin and ddGTP-Linker III-PC-Biotin are shown in FIG. 9. These compounds are synthesized by a similar chemistry as shown for the synthesis of ddATP-Linker II-11-Biotin in FIG. 6. The only difference is that in the final coupling step NHS-PC-LC-Biotin (Pierce, Rockford Ill.) is used, as shown in FIG. 10. The photocleavable linkers disclosed here allow the use of solid phase capturable terminators and mass spectrometry to be turned into a high throughput sequencing technique.

IV. Overview of Capturing a DNA Fragment Terminated With a ddNTP on a Surface and Freeing the ddNTP and DNA Fragment

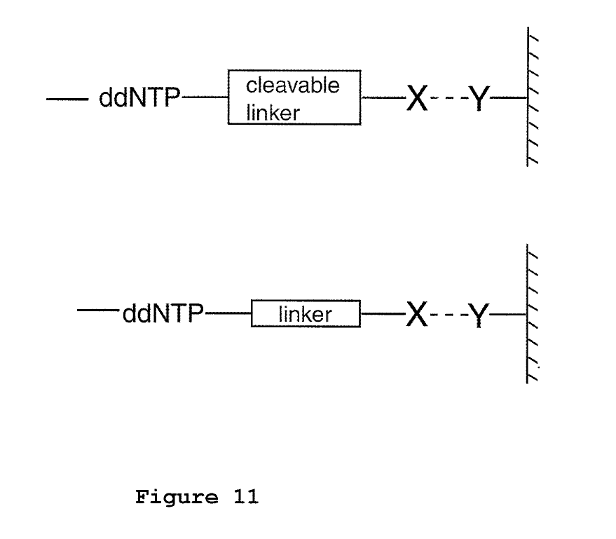

The DNA fragment is terminated with a dideoxynucleotide (ddNTP). The ddNTP is attached via a linker to a chemical moiety (“X” in FIG. 11). The dideoxynucleotide and DNA fragment are captured on the surface through interaction between chemical moiety “X” and a compound on or attached to the surface (“Y” in FIG. 11). The present application discloses two methods for freeing the captured dideoxynucleotide and DNA fragment. In the situation illustrated in the lower part of FIG. 11, the dideoxynucleotide and DNA fragment are freed from the surface by disrupting or breaking the interaction between chemical moiety “X” and compound “Y”. In the upper part of FIG. 11, the dideoxynucleotide is attached to chemical moiety “X” via a cleavable linker which can be cleaved to free the dideoxynucleotide and DNA fragment.

Different moieties and compounds can be used for the “X”-“Y” affinity system, which include but are not limited to, biotin-streptavidin, phenylboronic acid-salicylhydroxamic acid (Bergseid et al. 2000), and antigen-antibody systems.

In different embodiments, the cleavable linker can be cleaved and the “X”-“Y” interaction can be disrupted by a means selected from the group consisting of one or more of a physical means, a chemical means, a physical chemical means, heat, and light. In one embodiment, ultraviolet light can be used to cleave the cleavable linker. Chemical means include, but are not limited to, ammonium hydroxide (Jurinke et. al., 1997), formamide, or a change in pH (−log H⁺ concentration) of the solution.

V. High Density Streptavidin-Coated, Porous Silica Channel System.

Streptavidin coated magnetic beads are not ideal for using the photocleavable biotin capture and release process for DNA sequencing fragments, since they are not transparent to UV light. Therefore, the photocleavage reaction is not efficient. For efficient capture of the biotinylated sequencing fragments, a high-density surface coated with streptavidin is essential. It is known that the commercially available 96-well streptavidin coated plates cannot provide a sufficient surface area for efficient capture of the biotinylated DNA fragments. Disclosed in this application is a new porous silica channel system designed to overcome this limitation.

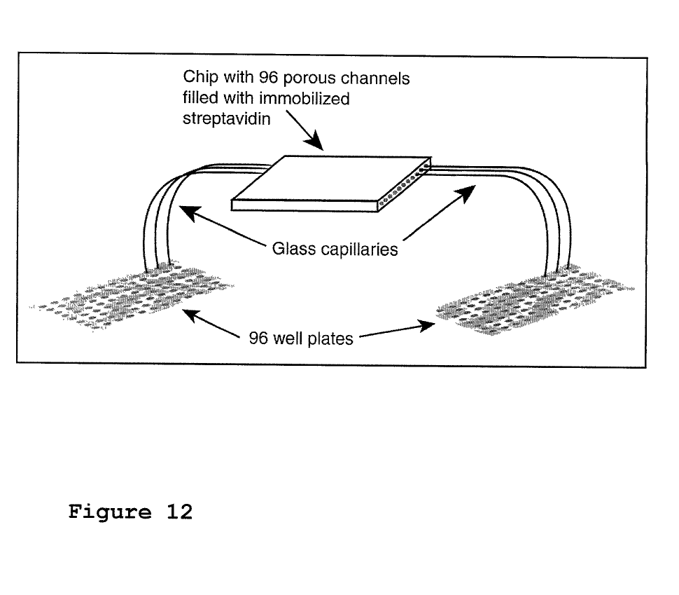

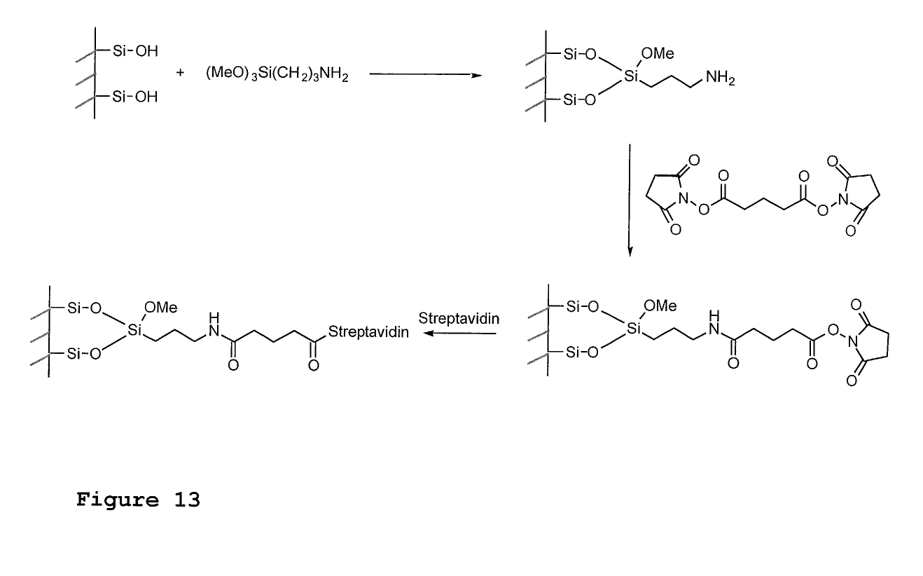

To increase the surface area available for solid phase capture, porous channels are coated with a high density of streptavidin. Ninety-six (96) porous silica glass channels can be etched into a silica chip (FIG. 12). The surfaces of the channels are modified to contain streptavidin as shown in FIG. 13. The channel is first treated with 0.5 M NaOH, washed with water, and then briefly pre-etched with dilute hydrogen fluoride. Upon cleaning with water, the capillary channel is coated with high density 3-aminopropyltrimethoxysilane in aqueous ethanol (Woolley et al. 1994). An excess of disuccinimidyl glutarate in N,N-dimethylformamide (DMF) is then introduced into the capillary to ensure a highly efficient conversion of the surface end group to a succinimidyl ester. Streptavidin is then conjugated with the succinimidyl ester to form a high-density surface using excess streptavidin solution. The resulting 96-channel chip is used as a purification cassette.

This application discloses a 96-well plate that can be used for sequencing fragment generation with biotinylated terminators as shown in FIG. 12. In the example shown, each end of a channel is connected to a single well. However, for other applications, the end of a channel could be connected to a plurality of wells. Pressure is applied to drive the samples through a glass capillary into the channels on the chip. Inside the channels the biotin is captured by the covalently bound streptavidin. After passing through the channel, the sample enters into a clean plate in the other end of the chip. Pressure applied in reverse drives the sample through the channel multiple times and ensures a highly efficient solid phase capture. Water is similarly added to drive out the reaction mixture and thoroughly wash the captured fragments. After washing, the chip is irradiated with ultraviolet light to cleave the photosensitive linker and release the DNA fragments.

The fragment solution is then driven out of the channel and into a collection plate. After matrix solution is added, the samples are spotted on a chip and allowed to crystallize for detection by MALDI-TOF mass spectrometry. The purification cassette is cleaned by chemically cleaving the biotin-streptavidin linkage, and is then washed and reused.

VI. Validation of the Mass Spectrometry DNA Sequencing System Using Synthetic DNA Templates and PCR Templates Generated from Genomic DNA.

To validate the sequencing technology disclosed here, a synthetic DNA template can be synthesized which mimics a portion of the human immunodeficiency virus type 1 protease gene. The sequence of the template (SEQ ID NO: 3) and that of the sequencing primer (SEQ ID NO: 4) are shown below (Schmit et al. 1996):

```
5′-TAAAGCTATAGGTACAGTATTAGTAGGACCTACACCTGTCAACATAATGGTCCAGGTCGTG-3′
        Template  ||||||||||||
                                                                                          3′-CCAGGTCCAGCAC-5′
                                                                                                      Primer
```

The tumor suppressor gene p53 can also be used as a model system. The p53 gene is one of the most frequently mutated genes in human cancer (O'Connor et al. 1997). Since most of the p53 mutation hot spots are clustered within exons 5-8, this region of the p53 gene is selected as a sequencing target. A synthetic sequencing template containing a portion of the sequences from exon 7 and exon 8 of the p53 gene and an appropriate primer can be prepared:

| | | |
|---|---|---|
| Template: | 5′-CATGTGAACAGTTCCTGCATGGGCGGCATGAACCCGAGG | (SEQ ID NO:5), |
| | CCCATCCTCACCATCATCACACTGGAAGACTCCAGTGGTAATCTACTGGGACG | |
| | GAACAGCTTTGAGGTGCATGTTTGTGCCTGTCCTGG-3′ | |
| Sequencing primer: | 5′-CCAGGACAGGCACAA-3′ | (SEQ ID NO:6). |

This template (SEQ ID NO: 5) was chosen to explore the use of the mass spectrometry sequencing procedure disclosed herein for the detection of clustered hot spot single base mutations. The potentially mutated bases are underlined (A, G, C and T) in the synthetic template shown above.

In addition to synthetic templates, DNA templates generated by polymerase chain reaction (PCR) can also be used to further validate the high fidelity MALDI-TOF mass spectrometry sequencing technology. The sequencing templates are generated by PCR using flanking primers in the intron region located at each p53 exon boundary from a pool of genomic DNA (Boehringer, Indianapolis, Ind.) as described by Fu et al. (1998).

### REFERENCES

Bergseid M, Baytan A R, Wiley J P, Ankener W M, Stolowitz, Hughs K A, Chestnut J D (Nov. 2000) Small-molecule base chemical affinity system for the purification of proteins. BioTechniques 29: 1126-1133.

Bowling J M, Bruner K L, Cmarik J L, Tibbetts C. (1991) Neighboring nucleotide interactions during DNA sequencing gel electrophoresis. Nucleic Acids Res. 19: 3089-3097.

Burgess K, Cook D. (2000) Chemical Reviews. 100: 2047-2060.

Chee M, Yang R, Hubbell E, Berno, A, Huang, X C., Stern D, Winkler, J, Lockhart D J, Morris M S, Fodor, S P. (1996) Accessing genetic information with high-density DNA arrays. Science 274: 610-614.

Chiu N H, Tang K, Yip P, Braun A, Koster H, Cantor C R. (2000) Mass spectrometry of single-stranded restriction fragments captured by an undigested complementary sequence. Nucleic Acids Res. 28: E31.

Fei Z, Ono T, Smith L M. (1998) MALDI-TOF mass spectrometric typing of single nucleotide polymorphisms with mass-tagged ddNTPs. Nucleic Acids Res. 26: 2827-2828.

Fu D J, Tang K, Braun A, Reuter D, Darnhofer-Demar B, Little D P, O'Donnell M J, Cantor C R, Koster H. (1998) Sequencing exons 5 to 8 of the p53 gene by MALDI-TOF mass spectrometry. Nat Biotechnol. 16: 381-384.

Gut I G, Beck S. (1995) A procedure for selective DNA alkylation and detection by mass spectrometry. Nucleic Acids Res. 23: 1367-1373.

Hobbs F W Jr, Cocuzza A J. Alkynylamino-Nucleotides. U.S. Pat. No. 5,047,519, issued Sep. 10, 1991.

Ju J, Ruan C, Fuller C W, Glazer A N Mathies R A. (1995) Energy transfer fluorescent dye-labeled primers for DNA sequencing and analysis. Proc. Natl. Acad. Sci. USA 92: 4347-4351.

Ju J, Glazer A N, Mathies R A. (1996) Cassette labeling for facile construction of energy transfer fluorescent primers. Nucleic Acids Res. 24: 1144-1148.

Ju J. Nucleic Acid Sequencing with Solid Phase Capturable Terminators. U.S. Pat. No. 5,876,936, issued Mar. 2, 1999.

Ju J, Konrad K. Nucleic Acid Sequencing with Solid Phase Capturable Terminators Comprising a Cleavable Linking Group. U.S. Pat. No. 6,046,005, issued Apr. 4, 2000.

Jurinke C, van de Boom D, Collazo V, Luchow A, Jacob A, Koster H. (1997) Recovery of nucleic acids from immobilized biotin-streptavidin complexes using ammonium hydroxide and applications in MALDI-TOF mass spectrometry. Anal. Chem. 69: 904-910.

Kheterpal I, Scherer J, Clark S M, Radhakrishnan A, Ju J, Ginther C L, Sensabaugh G F, Mathies R A. (1996) DNA Sequencing Using a Four-Color Confocal Fluorescence Capillary Array Scanner. Electrophoresis 17: 1852-1859.

Langer P R, Waldrop A A, Ward D C. (1981) Enzymatic synthesis of biotin-labeled polynucleotides: novel nucleic acid affinity probes. Proc. Natl. Acad. Sci. USA. 78: 6633-6637.

Lee L G, Connell C R, Woo S L, Cheng R D, Mcardle B F, Fuller C W, Halloran N D, Wilson R K. (1992) DNA sequencing with dye-labeled terminators and T7 DNA-polymerase-effect of dyes and dNTPs on incorporation of dye-terminators and probability analysis of termination fragments. Nucleic Acids Res. 20: 2471-2483.

Maudling D R, Lotts K D, Robinson S A. (1983) New procedure for making 2-(chloromethyl)-4-nitrotoluene. J. Org. Chem. 48: 2938.

Monforte J A, Becker C H (1997) High-throughput DNA analysis by time-of-flight mass spectrometry. Nat Med. 3(3): 360-362.

O'Connor P M, Jackman J, Bae I, Myers T G, Fan S, Mutoh M, Scudiero D A, Monks A, Sausville E A, Weinstein J N, Friend S, Fornace A J Jr, Kohn K W. (1997) Characterization of the p53 tumor suppressor pathway in cell lines of the National Cancer Institute anticancer drug screen and correlations with the growth-inhibitory potency of 123 anticancer agents. Cancer Res. 57: 4285-4300.

Olejnik J, Sonar S, Krzymanska-Olejnik E, Rothschild K J. (1995) Photocleavable biotin derivatives: a versatile approach for the isolation of biomolecules. Proc. Natl. Acad. Sci. USA. 92: 7590-7594.

Olejnik J, Ludemann H C, Krzymanska-Olejnik E, Berkenkamp S, Hillenkamp F, Rothschild KJ. (1999) Photocleavable peptide-DNA conjugates: synthesis and applications to DNA analysis using MALDI-MS. Nucleic Acids Res. 27: 4626-4631.

Ono T, Scalf M, Smith L M. (1997) 2′-Fluoro modified nucleic acids: polymerase-directed synthesis, properties and stability to analysis by matrix-assisted laser desorption/ionization mass spectrometry. Nucleic Acids Res. 25: 4581-4588.

Pennisi E. (2000) DOE Team Sequences Three Chromosomes. Science 288: 417-419.

Prober J M, Trainor G L, Dam R J, Hobbs F W, Robert son C W, Zagursky R J, Cocuzza A J, Jensen M A, Baumeister K. (1987) A system for rapid DNA sequencing with fluorescent chain-terminating dideoxynucleotides. Science 238: 336-341.

Rolla F. (1982) Sodium-borohydride reactions under phase-transfer conditions—reduction of azides to amines. J. Org. Chem. 47: 4327-4329.

Rosenblum B B, Lee L G, Spurgeon S L, Khan S H, Menchen S M, Heiner C R, Chen S M. (1997) New dye-labeled terminators for improved DNA sequencing patterns. Nucleic Acids Res. 25: 4500-4504.

Roses A. (2000) Pharmacogenetics and the practice of medicine. Nature. 405: 857-865.

Roskey M T, Juhasz P, Smirnov I P, Takach E J, Martin S A, Haff L A. (1996) DNA sequencing by delayed extraction-matrix-assisted laser desorption/ionization time of flight mass spectrometry. Proc. Natl. Acad. Sci. USA. 93: 4724-4729.

Salas-Solano O, Carrilho E, Kotler L, Miller A W, Goetzinger W, Sosic Z, Karger B L, (1998) Routine DNA sequencing of 1000 bases in less than one hour by capillary electrophoresis with replaceable linear polyacrylamide solutions. Anal. Chem. 70: 3996-4003.

Sanger F, Nickeln S and Coulson A R (1977) DNA sequencing with chain-terminating inhibitors. Proc. Natl. Acad. Sci. USA 74: 5463-5467

Schmit J. C, Ruiz L, Clotet B, Raventos A, Tor J, Leonard J, Desmyter J. De Clercq E, Vandamme A M. (1996) Resistance-related mutations in the HIV-1 protease gene of patients treated for 1 year with the protease inhibitor ritonavir (ABT-538) AIDS 10: 995-999.

Schneider K, Chait B T. (1995) Increased stability of nucleic acids containing 7-deaza-guanosine and 7-deaza-adenosine may enable rapid DNA sequencing by matrix-assisted laser desorption mass spectrometry. Nucleic Acids Res. 23: 1570-1575.

Smith L M, Sanders J Z, Kaiser R J, Hughes P, Dodd C, Connell C R, Heiner C, Kent S B H, Hood L E. (1986) Fluorescence detection in automated DNA sequencing analysis. Nature 321: 674-679.

Tabor S, Richardson C. C. (1987) DNA sequence analysis with a modified bacteriophage T7 DNA polymerase. Proc. Natl. Acad. Sci. U.S.A. 84: 4767-4771.

Tabor S. & Richardson, C C. (1995) A single residue in DNA polymerases of the Escherichia coli DNA polymerase I family is critical for distinguishing between deoxy- and dideoxyribonucleotides. Proc. Natl. Acad. Sci. U.S.A. 92: 6339-6343.

Tang K, Fu D J, Julien D, Braun A, Cantor C R, Koster H. (1999) Chip-based genotyping by mass spectrometry. Proc. Natl. Acad. Sci. USA. 96: 10016-10020.

Tong, X., Smith L M (1992) Solid-Phase Method for the Purification of DNA Sequencing Reactions. Anal. Chem. 64: 2672-2677.

Tong, X., Smith L M (1993) Solid Phase Purification in Automated DNA Sequencing. DNA Sequence-J. DNA Sequencing and Mapping 4: 151-162.

Velculescu V E, Zhang, I, Vogelstein, B. and Kinzler K W (1995) Serial Analysis of Gene Expression. Science 270: 484-487.

Woolley A T, Mathies R A. (1994) Ultra-high-speed DNA fragment separations using microfabricated capillary array electrophoresis chips. Proc. Natl. Acad. Sci. USA. 91: 11348-11352.

Wu K J, Steding A, Becker C H. (1993) Matrix-assisted laser desorption time-of-flight mass spectrometry of oligonucleotides using 3-hydroxypicolinic acid as an ultraviolet-sensitive matrix. Rapid Commun Mass Spectrom. 7: 142-146.

Xu L, Bian N, Wang Z, Abdel-Baky S, Pillai S, Magiera D, Murugaiah V, Giese R W, Wang P, O'Keeffe T, Abushamaa H, Kutney L, Church G, Carson S, Smith D, Park M, Wronka J, Laukien F. (1997) Electrophore mass tag dideoxy DNA sequencing. Anal. Chem. 69: 3595-3602.

Yamakawa H, Ohara O. (1997) A DNA cycle sequencing reaction that minimizes compressions on automated fluorescent sequencers. Nucleic. Acids. Res. 25: 1311-1312.

Zhu Z, Chao J, Yu H, Waggoner A S. (1994) Directly labeled DNA probes using fluorescent nucleotides with different length linkers. Nucleic Acids Res. 22: 3418-3422.

What is claimed is:

1. A method for sequencing DNA by detecting the identity of a dideoxynucleotide incorporated to the 3′ end of a DNA sequencing fragment using mass spectrometry, which comprises:
(a) attaching a chemical moiety via a linker to a dideoxynucleotide to produce a labeled dideoxynucleotide;
(b) terminating a DNA sequencing reaction with the labeled dideoxynucleotide to generate a labeled DNA sequencing fragment, wherein the DNA sequencing fragment has a 3′ end and the chemical moiety is attached via the linker to the 3′ end of the DNA sequencing fragment;
(c) capturing the labeled DNA sequencing fragment on a surface coated with a compound that specifically interacts with the chemical moiety attached via the linker to the DNA sequencing fragment, thereby capturing the DNA sequencing fragment;
(d) washing the surface to remove any non-bound component;
(e) freeing the DNA sequencing fragment from the surface; and
(f) analyzing the DNA sequencing fragment using mass spectrometry so as to sequence the DNA.

2. A method for sequencing DNA by detecting the identity of a plurality of dideoxynucleotides incorporated to the 3′ end of different DNA sequencing fragments using mass spectrometry, which comprises:
(a) attaching a chemical moiety via a linker to a plurality of different dideoxynucleotides to produce labeled dideoxynucleotides;
(b) terminating a DNA sequencing reaction with the labeled dideoxynucleotides to generate labeled DNA sequencing fragments, wherein the DNA sequencing fragments have a 3′ end and the chemical moiety is attached via the linker to the 3′ end of the DNA sequencing fragments;
(c) capturing the labeled DNA sequencing fragments on a surface coated with a compound that specifically interacts with the chemical moiety attached via the linker to the DNA sequencing fragments, thereby capturing the DNA sequencing fragments;
(d) washing the surface to remove any non-bound component;
(e) freeing the DNA sequencing fragments from the surface; and
(f) analyzing the DNA sequencing fragments using mass spectrometry so as to sequence the DNA.

3. The method of claim 2, wherein the chemical moiety is attached via a different linker to different dideoxynucleotides.

4. The method of claim 1 or 2, wherein the interaction between the chemical moiety attached via the linker to the DNA sequencing fragment and the compound on the surface comprises a biotin-streptavidin interaction, a phenylboronic acid-salicylhydroxamic acid interaction, or an antigen-antibody interaction.

5. The method of claim 1 or 2, wherein the step of freeing the DNA sequencing fragment from the surface comprises disrupting the interaction between the chemical moiety attached via the linker to the DNA sequencing fragment and the compound on the surface.

6. The method of claim 5, wherein the interaction is disrupted by a means selected from the group consisting of one or more of a physical means, a chemical means, a physical chemical means, heat, and light.

7. The method of claim 1 or 2, wherein the dideoxynucleotide comprises a cytosine or a thymine with a 5-position, or an adenine or a guanine with a 7-position, and the linker is attached to the 5-position of cytosine or thymine or to the 7-position of adenine or guanine.

8. The method of claim 1 or 2, wherein the step of freeing the DNA sequencing fragment from the surface comprises cleaving the linker.

9. The method of claim 8, where the linker is cleaved by a means selected from the group consisting of one or more of a physical means, a chemical means, a physical chemical means, heat, and light.

10. The method of claim 9, wherein the linker is cleaved by ultraviolet light.

11. The method of claim 1 or 2, wherein the linker comprises a derivative of 4-aminomethyl benzoic acid.

12. The method of claim 11, wherein the linker comprises one or more fluorine atoms.

13. The method of claim 12, wherein the linker is selected from the group consisting of:

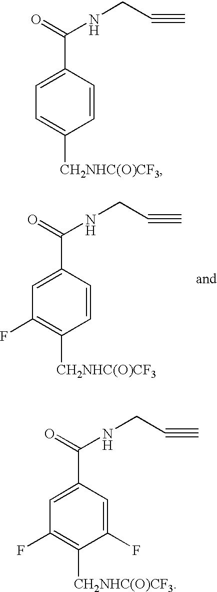

14. The method of claim 1, wherein a plurality of different labeled dideoxynucleotides is used to generate a plurality of different labeled DNA sequencing fragments.

15. The method of claim 3 or 14, wherein a plurality of different linkers is used to increase mass separation between different labeled DNA sequencing fragments and thereby increase mass spectrometry resolution.

16. The method of claim 1 or 2, wherein the chemical moiety comprises biotin, the labeled dideoxynucleotide is a biotinylated dideoxynucleotide, the labeled DNA sequencing fragment is a biotinylated DNA sequencing fragment, and the surface is a streptavidin-coated solid surface.

17. The method of claim 16, wherein the biotinylated dideoxynucleotide is selected from the group consisting of ddATP-11-biotin, ddCTP-11-biotin, ddGTP-11-biotin, and ddTTP-16-biotin.

18. The method of claim 16, wherein the biotinylated dideoxynucleotide is selected from the group consisting of: 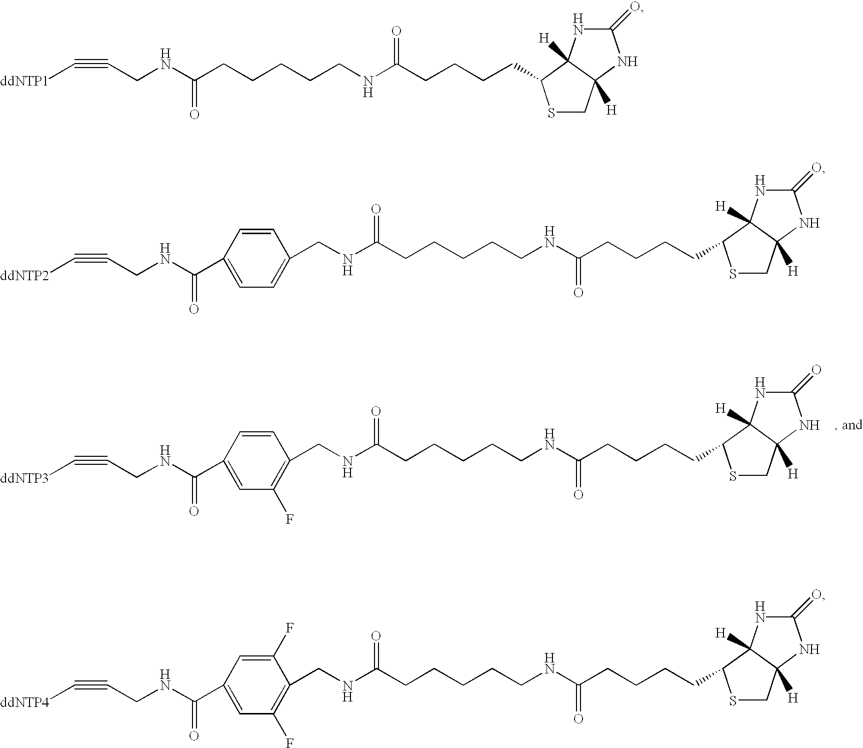
wherein ddNTP1, ddNTP2, ddNTP3, and ddNTP4 represent four different dideoxynucleotides.

19. The method of claim 18, wherein the biotinylated dideoxynucleotide is selected from the group consisting of:

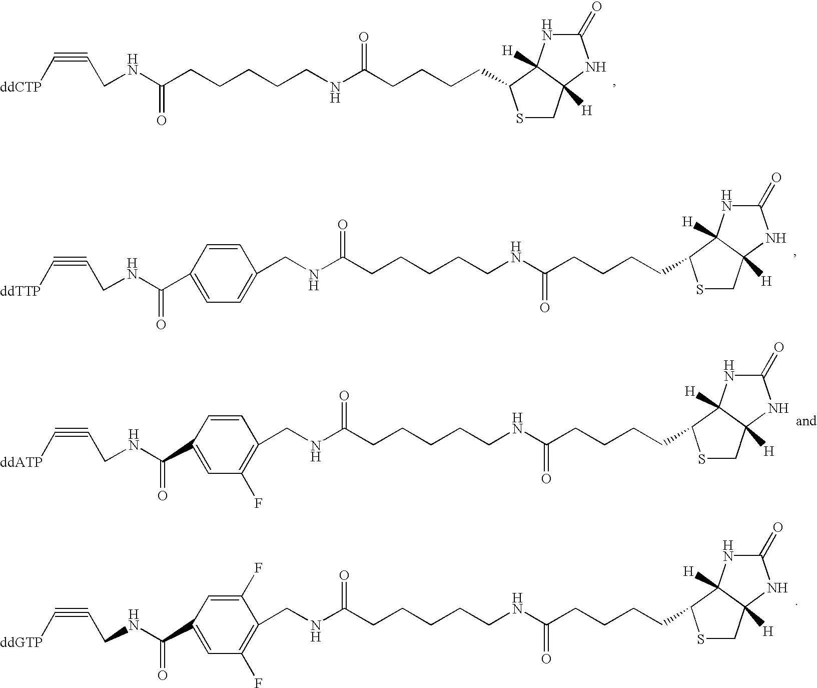

20. The method of claim 16, wherein the biotinylated dideoxynucleotide is selected from the group consisting of: 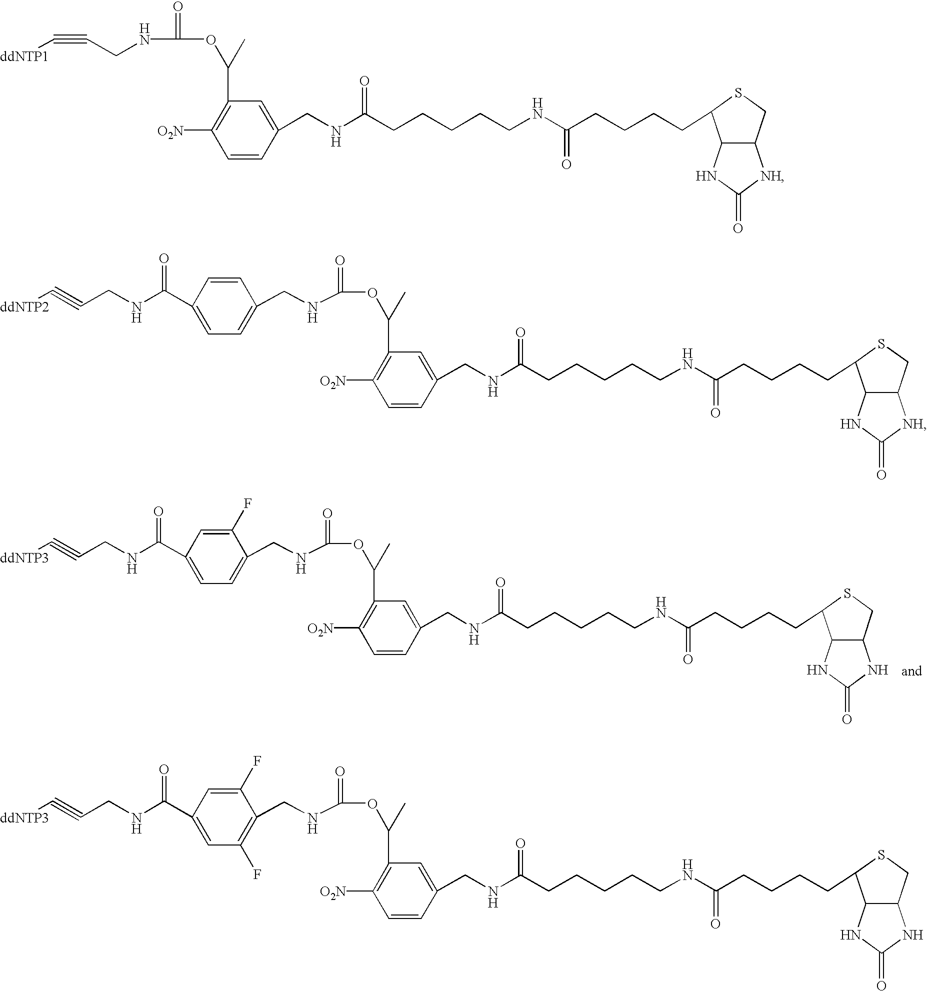
wherein ddNTP1, ddNTP2, ddNTP3, and ddNTP4 represent four different dideoxynucleotides.

21. The method of claim 20, wherein the biotinylated dideoxynucleotide is selected from the group consisting of:

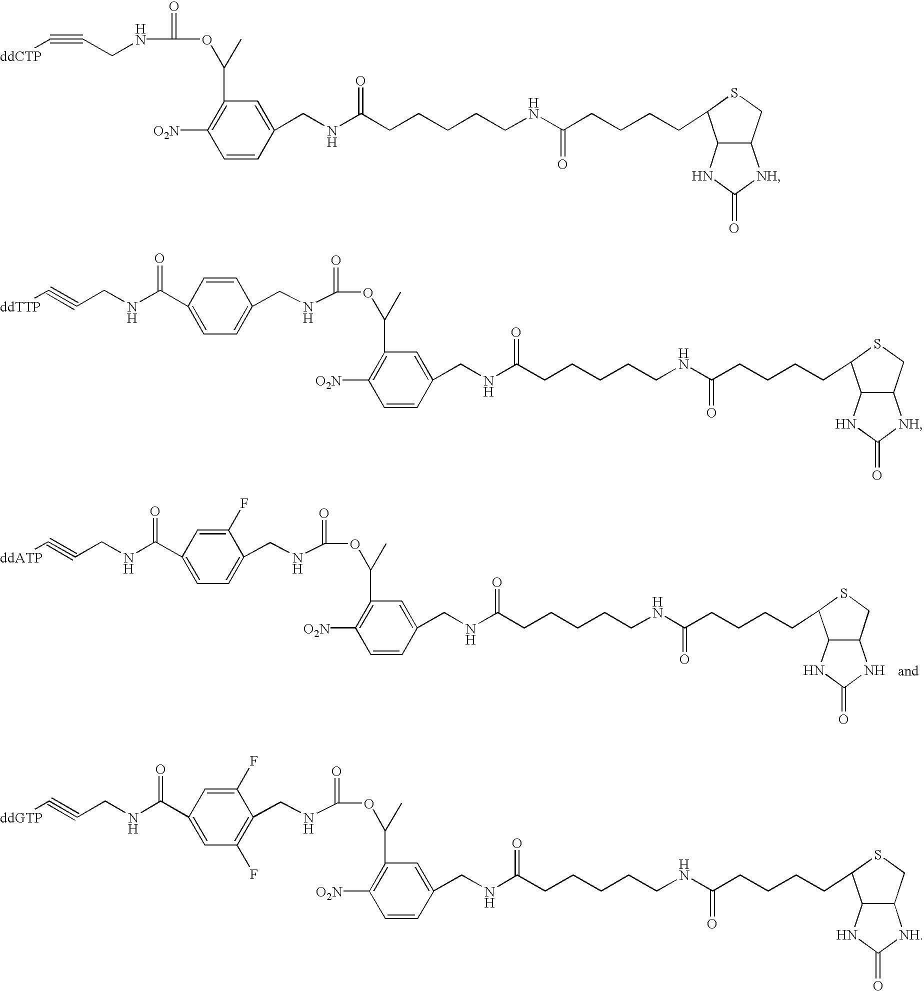

22. The method of claim 16, wherein the streptavidin-coated solid surface is a streptavidin-coated magnetic bead or a streptavidin-coated silica glass.

23. The method of claim 1 or 2, wherein steps (b) to (e) are performed in a single container or in a plurality of connected containers.

24. Use of the method of claim 1 or 2 for detection of single nucleotide polymorphisms, genetic mutation analysis, serial analysis of gene expression, gene expression analysis, identification in forensics, genetic disease association studies, genomic sequencing, translational analysis, or transcriptional analysis.

25. A linker for attaching a chemical moiety to a dideoxynucleotide, wherein the linker comprises a derivative of 4-aminomethyl benzoic acid.

26. The linker of claim 25, wherein the linker comprises one or more fluorine atoms.

27. The linker of claim 26, wherein the linker is selected from the group consisting of:

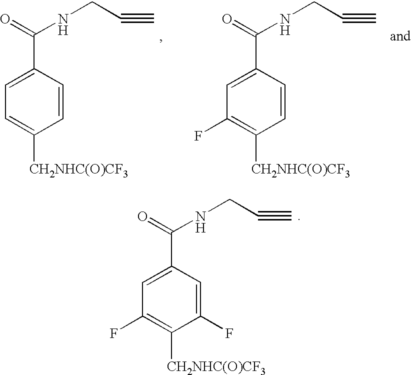

28. The linker of claim 25, wherein the linker is cleavable by a means selected from the group consisting of one or more of a physical means, a chemical means, a physical chemical means, heat, and light.

29. The linker of claim 28, wherein the linker is cleavable by ultraviolet light.

30. The linker of claim 25, wherein the chemical moiety comprises biotin, streptavidin, phenylboronic acid, salicylhydroxamic acid, an antibody, or an antigen.

31. The linker of claim 25, wherein the dideoxynucleotide comprises a cytosine or a thymine with a 5-position, or an adenine or a guanine with a 7-position, and the linker is attached to the 5-position of cytosine or thymine or to the 7-position of adenine or guanine.

32. Use of the linker of claim 25 in DNA sequencing using mass spectrometry, wherein the linker increases mass separation between different dideoxynucleotides and increases mass spectrometry resolution.

33. A labeled dideoxynucleotide, which comprises a chemical moiety attached via a linker to a 5-position of cytosine or thymine or to a 7-position of adenine or guanine.

34. The labeled dideoxynucleotide of claim 33, wherein the linker is cleavable by a means selected from the group consisting of one or more of a physical means, a chemical means, a physical chemical means, heat, and light.

35. The labeled dideoxynucleotide of claim 34, wherein the linker is cleavable by ultraviolet light.

36. The labeled dideoxynucleotide of claim 33, wherein the chemical moiety comprises biotin, streptavidin, phenylboronic acid, salicylhydroxamic acid, an antibody, or an antigen.

37. The labeled dideoxynucleotide of claim 33, wherein the labeled dideoxynucleotide is selected from the group consisting of: 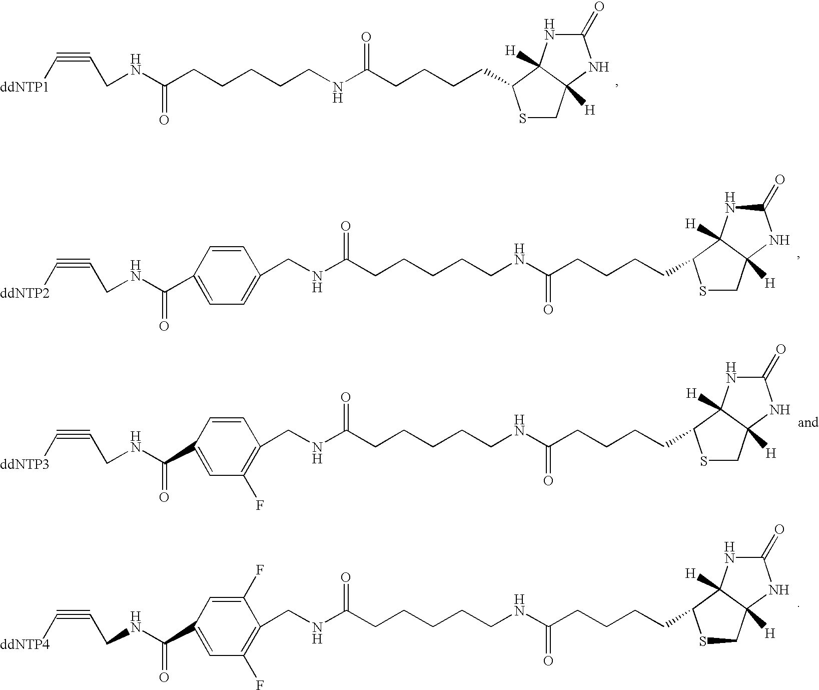
wherein ddNTP1, ddNTP2, ddNTP3, and ddNTP4 represent four different dideoxynucleotides.

38. The labeled dideoxynucleotide of claim 37, wherein the labeled dideoxynucleotide is selected from the group consisting of:

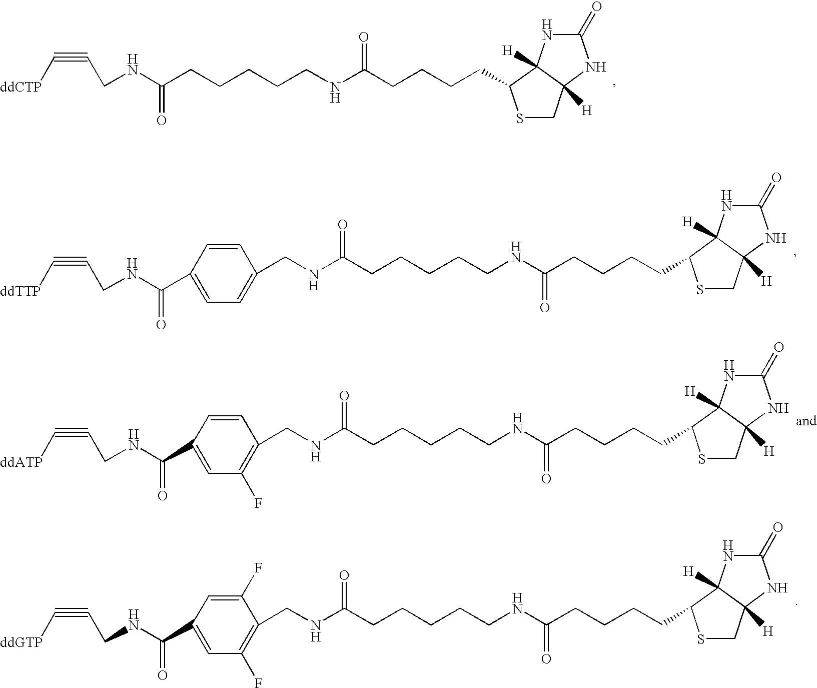

39. The labeled dideoxynucleotide of claim 33, wherein the labeled dideoxynucleotide is selected from the group consisting of: 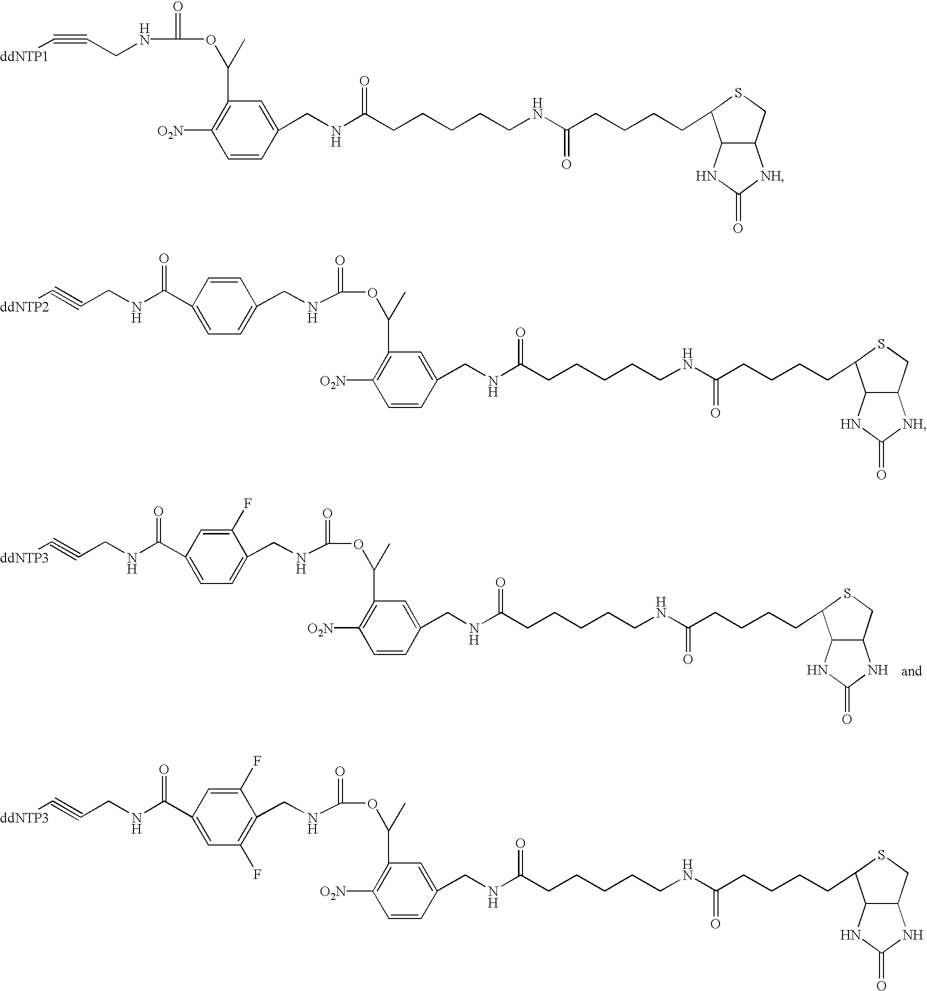
wherein ddNTP1, ddNTP2, ddNTP3, and ddNTP4 represent four different dideoxynucleotides.

40. The labeled dideoxynucleotide of claim 39, wherein the labeled dideoxynucleotide is selected from the group consisting of:

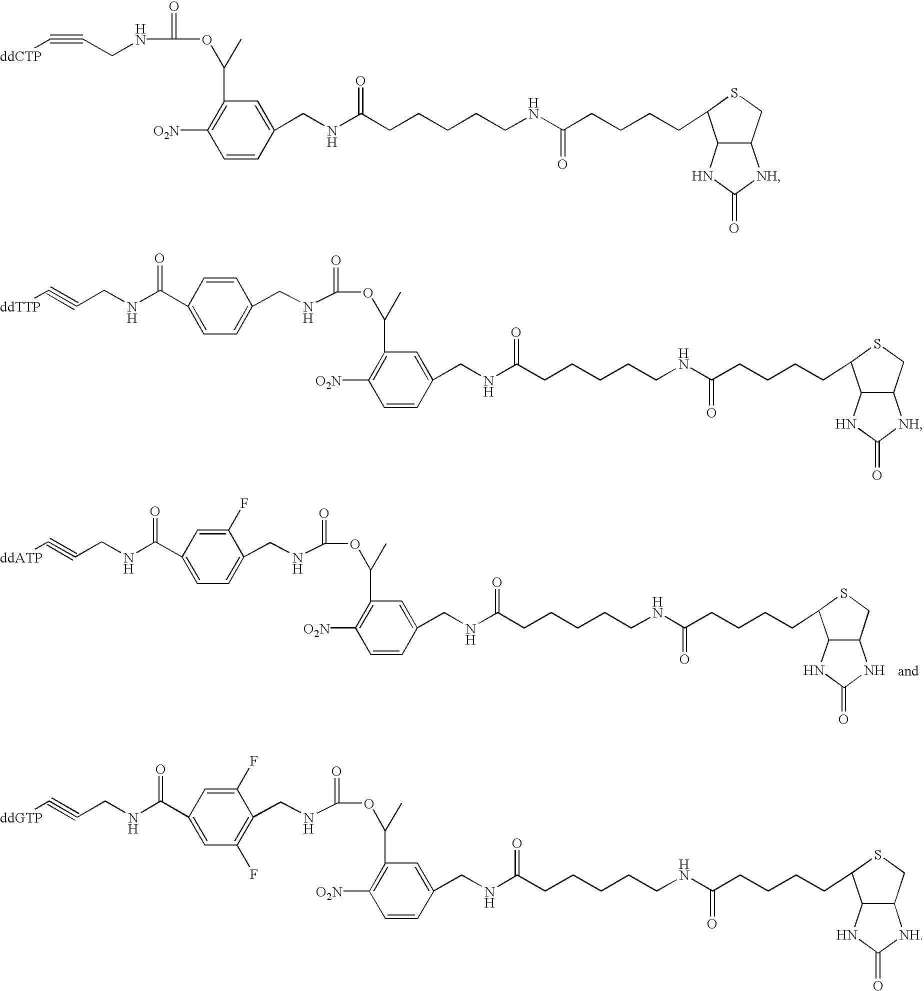

41. Use of the labeled dideoxynucleotide of claim 33 in DNA sequencing using mass spectrometry, wherein the linker increases mass separation between different labeled dideoxynucleotides and increases mass spectrometry resolution.

42. A system for separating a chemical moiety. from other components in a sample in solution, which comprises:
(a) a channel coated with a compound that specifically interacts with the chemical moiety, wherein the channel comprises a plurality of ends;
(b) a plurality of wells each suitable for holding the sample;
(c) a connection between each end of the channel and a well; and
(d) a means for moving the sample through the channel between wells.

43. The system of claim 42, wherein the interaction between the chemical moiety and the compound coating the surface is a biotin-streptavidin interaction, a phenylboronic acid-salicylhydroxamic acid interaction, or an antigen-antibody interaction.

44. The system of claim 42, wherein the chemical moiety is a biotinylated moiety and the channel is a streptavidin-coated silica glass channel.

45. The system of claim 44, wherein the biotinylated moiety is a biotinylated DNA sequencing fragment.

46. The system of claim 42, wherein the chemical moiety can be freed from the surface by disrupting the interaction between the chemical moiety and the compound coating the surface.

47. The system of claim 46, where the interaction can be disrupted by a means selected from the group consisting of one or more of a physical means, a chemical means, a physical chemical means, heat, and light.

48. The system of claim 42, wherein the chemical moiety is attached via a linker to another chemical compound.

49. The system of claim 48, wherein the other chemical compound is a DNA sequencing fragment.

50. The system of claim 48, where the linker is cleavable by a means selected from the group consisting of one or more of a physical means, a chemical means, a physical chemical means, heat, and light.

51. The system of claim 50, wherein the channel is transparent to ultraviolet light and the linker is cleavable by ultraviolet light.

52. A multi-channel system, which comprises a plurality of the system of claim 42.

53. The multi-channel system of claim 52, wherein the channels are in a chip.

54. The multi-channel system of claim 53, which comprises 96 channels in a chip.

55. Use of the system of claim 42 or 52 for separating one or more DNA sequencing fragments, wherein each fragment is terminated with a dideoxynucleotide attached via a linker to the chemical moiety.

56. A method of increasing mass spectrometry resolution between different DNA sequencing fragments, which comprises attaching different linkers to different dideoxynucleotides used to terminate a DNA sequencing reaction and generate different DNA sequencing fragments, wherein the different linkers increase mass separation between the different DNA sequencing fragments, thereby increasing mass spectrometry resolution.

57. The method of claim 56, wherein one or more of the different linkers comprises one or more fluorine atoms.

58. The method of claim 57, wherein one or more of the different linkers is selected from the group consisting of: [chemical structure]
